(19) United States
(12) Patent Application Publication
(10) Pub. No.: US 2018/0153970 A1
(43) Pub. Date: Jun. 7, 2018

(54) COMPOSITION AND USES THEREOF

(71) Applicant: Arborvitae Health and Wellbeing Pty. Ltd., New South Wales (AU)

(72) Inventor: Victor Patrick DAVIDSON, New South Wales (AU)

(73) Assignee: Arborvitae Health and Wellbeing Pty. Ltd., New South Wales (AU)

(21) Appl. No.: 15/576,416

(22) PCT Filed: Jul. 14, 2015
(86) PCT No.: PCT/AU2015/000406

§ 371 Date: Nov. 22, 2017

(30) Foreign Application Priority Data
May 25, 2015 (AU) 2015901913

(51) Int. Cl. A61K 38/48; A61K 36/15; A61K 36/886; A61K 47/12; A61K 47/46; A61K 47/02; A61K 9/00; A61P 3/10; A61P 9/10; A61P 1/16; A61P 3/04; A61P 15/00; A61P 19/02; A61K 31/155; A61P 9/00; A61P 37/02; A61P 17/06

(52) U.S. Cl. CPC A61K 38/4873; A61P 17/06; C12Y 304/22002; A61K 36/886; A61K 47/12; A61K 47/46; A61K 47/02; A61K 9/0053; A61P 3/10; A61P 9/10; A61P 1/16; A61P 3/04; A61P 15/00; A61P 19/02; A61K 31/155; A61P 9/00; A61P 37/02; A61K 36/15

(57) ABSTRACT

The invention relates to compositions comprising Pinus pinaster stem bark extract, papain, and Aloe vera extract, and methods of manufacturing same. There are also described methods of treating or preventing a variety of conditions, including treating or preventing elevated blood glucose, pre-diabetes, type 2 diabetes, autoimmune diseases, reducing or decreasing inflammation, treating or preventing diseases characterised by elevated levels of inflammation, and lowering blood cholesterol, said methods comprising administering an effective amount of a composition according to the invention to a subject in need thereof. Uses of the composition of the invention for the manufacture of a medicament for treating or preventing a variety of conditions are also described.

### TECHNICAL FIELD

The present invention relates generally to the field of health supplements. More specifically, the present invention relates to a composition comprising Pinus pinaster stem bark extract, papain and Aloe vera extract and to its use in improving health, for example, regulating blood sugar levels and treating, delaying or preventing conditions associated with or caused by elevated blood sugar levels.

### PRIORITY

The present application claims priority from Australian provisional application AU 2015901913, the entire contents of which are incorporated herein by cross-reference.

### BACKGROUND

### Pre-Diabetes and Diabetes

According to Diabetes Australia, nearly one in four adults over the age of 25 years has either diabetes or a condition known as ‘pre-diabetes’. Pre-diabetes is a condition in which blood glucose levels are higher than normal, but not high enough to be diagnosed as type 2 diabetes mellitus (T2DM). Pre-diabetes is a condition in which blood glucose levels are higher than normal, although not high enough to be classified as diabetes. Pre-diabetes may have no signs or symptoms. People diagnosed with pre-diabetes have a higher risk of developing T2DM and cardiovascular (heart and circulation) disease. Pre-diabetes is diagnosed by a blood test that checks the blood glucose level. Any blood glucose test that shows higher than normal blood glucose levels needs to be followed up by a physician. This is likely to involve an oral glucose tolerance test (OGTT). The results of the OGTT will show whether the blood glucose levels are in the normal, pre-diabetes or diabetes range. If a person is diagnosed with pre-diabetes, they will have one or both of the following
impaired fasting glucose (IFG)—this is when the fasting blood glucose level is higher [in the range of 6.1 mmol/L to 6.9 mmol/L] than normal, but still below the level for a diagnosis of diabetes.
impaired glucose tolerance (IGT)—is when the blood glucose level two hours after an OGTT is higher [in the range of 7.8 to 11.0 mmol/L] than normal, but is still below the level for a diagnosis of diabetes. The fasting blood glucose level may be in the normal range.
   Sometimes the oral diabetes drug metformin (Glucophage) may be recommended by a doctor if an individual is at high risk of progressing to T2DM. This includes people whose body mass index is over 35, people who are younger than 60 years and women with a history of gestational diabetes.

### Type 2 Diabetes

Islet dysfunction and peripheral insulin resistance are both present in T2DM and are both necessary for the development of hyperglycemia. In both type I diabetes mellitus (T1DM) and T2DM, large, prospective clinical studies have shown a strong relation between time-averaged mean values of glycemia, measured as glycated hemoglobin (HbA1c), and vascular diabetic complications. These studies are the basis for the American Diabetes Association's current recommended treatment goal that HbA1c should be <7%. The measurement of the HbA1c concentration is considered the gold standard for assessing long-term glycemia. However, it does not reveal any information on the extent or frequency of blood glucose excursions, but provides an overall mean value only. Postprandial hyperglycemia occurs frequently in patients diagnosed with T2DM receiving active treatment and can occur even when metabolic control is apparently good. Interventional studies indicate that reducing post-meal glucose excursions is as important as controlling fasting plasma glucose in persons with T2DM and impaired glucose tolerance. Evidence exists for a causal relation between post-meal glucose increases and microvascular and macrovascular outcomes. Therefore, treatments with different compounds that have specific effects on postprandial glucose regulation is accompanied by a significant improvement of many pathways supposed to be involved in diabetic complications, including endothelial dysfunction, inflammation, and nuclear factor-kappaB activation. The goal of therapy should be to achieve glycemic status as near to normal as safely possible in all 3 components of glycemic control, these including HbA1c, fasting glucose, and post-meal glucose peak. The overall adverse effect in failing to control these factors is to increase the risk of disease, in particular in the older age groups.

There is a need for new methods and compositions that provide one or more health benefits, such as improving overall health, improving organ function, treating and preventing elevated blood sugar related diseases such as T2DM and its related chronic diseases, treating and preventing conditions associated with or causative from elevated blood sugar such as pre-diabetes, treating inflammation and its associated diseases, and/or lowering blood cholesterol.

### SUMMARY OF THE INVENTION

The present inventor has identified a composition comprising Pinus pinaster stem bark extract, papain, and Aloe vera extract that exerts a number of health benefits, for example, the ability to improve organ function, reduce inflammation, and regulate blood sugar levels.

Accordingly, in a first aspect, the present invention provides a composition comprising Pinus pinaster stem bark extract, papain, and Aloe vera extract.

In a second aspect, the present invention provides a composition comprising a therapeutically effective amount of Pinus pinaster stem bark extract, papain, and Aloe vera extract.

In a third aspect, the present invention provides an aqueous composition comprising about 2.6 mg/mL Pinus pinaster stem bark extract, about 2.4 mg/mL papain, and about 1.75 mg/mL Aloe vera extract.

The following options may be used in conjunction with the first, second or third aspects above either individually or in any suitable combination.

The Aloe vera extract may be Aloe vera leaf extract.

The therapeutically effective amount of Pinus pinaster stem bark extract may correspond to a daily adult human dosage of from about 60 mg to about 1500 mg Finns pinaster stem bark extract; the therapeutically effective amount of papain may correspond to a daily adult human dosage of from about 30 mg to about 1200 mg papain; and the therapeutically effective amount of Aloe vera extract may correspond to a daily adult human dosage of from about 15 mg to about 600 mg Aloe vera extract.

In one embodiment, the therapeutically effective amount of Pinus pinaster stem bark extract corresponds to a daily adult human dosage of about 260 mg; the therapeutically effective amount of papain corresponds to a daily adult human dosage of about 240 mg; and the therapeutically effective amount of Aloe vera extract corresponds to a daily adult human dosage of about 175 mg.

The composition may further comprise an acid. The acid may be acetic acid. The acid may be present in a concentration effective to adjust the pH of the composition to between about 3.2 and about 4.2.

The composition may further comprise honey.

The composition may further comprise one or more additives selected from the group consisting of: a preservative, a flavouring agent, a salt, and a pigment. The composition may comprise a salt, and the salt may be sodium chloride.

The composition may comprise an additional component selected from the group consisting of: an omega-3 fatty acid, a phytonutrient, a source of protein, an amino acid, an antioxidant, a vitamin, a mineral, a plant extract, and mixtures thereof.

The composition may be synergistic.

The composition may be adapted for oral administration. For example, the composition may be in a form selected from the group consisting of: a tablet, a capsule, a chewable tablet, a soft gel capsule, a sachet, a powder, granules, a liquid, a syrup, a liquid suspension, an emulsion, a solution, and combinations thereof. The composition may be an aqueous composition.

The composition may have a pH of about 3.7.

The Pinus pinaster stem bark extract may be present at a concentration of from about 2 mg/mL to about 10 mg/mL. The Pinus pinaster stem bark extract may be present at a concentration of about 2.6 mg/mL.

The papain may be present at a concentration of from about 1 mg/mL to about 5 mg/mL. The papain may be present at a concentration of about 2.4 mg/mL.

The Aloe vera extract may be present at a concentration of from about 0.5 mg/mL to about 4 mg/mL. The Aloe vera extract may be present at a concentration of about 1.75 mg/mL.

The composition may comprise glacial acetic acid at a concentration of from about 1 mg/mL to about 8 mg/mL. The glacial acetic acid may be present at a concentration of about 2 mg/mL.

The composition may comprise honey at a concentration of from about 50 mg/mL to about 200 mg/mL. The honey may be present at a concentration of about 100 mg/mL.

In an embodiment, each mL of the composition comprises Pinus pinaster (Maritime Pine) stem bark concentrated extract equivalent to 1.56 g dry Pinus pinaster stem bark standardised to contain 1.82 mg procyanidins, 2.4 mg papain, 3 mg sodium chloride, and Aloe vera dried inner leaf juice equivalent to 350 mg fresh juice.

The composition may further comprise a source of omega-3 fatty acids selected from the group consisting of fish oil, krill, plant sources containing omega-3 fatty acids, flaxseed, walnut, algae, and combinations thereof. The omega-3 fatty acids may be selected from the group consisting of alpha-linolenic acid (“ALA”), docosahexaenoic acid (“DHA”), stearidonic acid (“SD”) and combinations thereof. The omega-3 fatty acids may be provided in an amount of about 0.25 g to 5.0 g per day.

The composition may further comprise a phytonutrient selected from the group consisting of flavanoids, allied phenolic compounds, polyphenolic compounds, terpenoids, alkaloids, sulfur-containing compounds, and combinations thereof. The phytonutrient may be selected from the group consisting of carotenoids, plant sterols, quercetin, curcumin, limonin, and combinations thereof.

The composition may comprise a source of protein. The source of protein may provide the composition with at least 10 g of high quality protein. The source of protein may be selected from the group consisting of dairy based proteins, plant based proteins, animal based proteins, artificial proteins, and combinations thereof. The dairy based proteins may be selected from the group consisting of casein, micellar casein, caseinates, casein hydrolysate, whey, whey protein micelles, whey hydrolysates, whey concentrates, whey isolates, milk protein concentrate, milk protein isolate, and combinations thereof. The plant based proteins may be selected from the group consisting of soy protein, pea protein, canola protein, wheat and fractionated wheat proteins, corn proteins, Zein proteins, rice proteins, oat proteins, potato proteins, peanut proteins, green pea powder, green bean powder, spirulina, proteins derived from vegetables, beans, buckwheat, lentils, pulses, single cell proteins, and combinations thereof.

The composition may further comprise an amino acid selected from the group consisting of alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, and combinations thereof. The amino acid may be a branched chain amino acid selected from the group consisting of isoleucine, leucine, valine, and combinations thereof.

The composition may further comprise an antioxidant selected from the group consisting of astaxanthin, carotenoids, coenzyme Q10 (“CoQ10”), flavonoids, glutathione, Goji (Wolfberry), hesperidin, lactowolfberry, lignan, lutein, lycopene, polyphenols, selenium, vitamin A, vitamin C, vitamin E, Zeaxanthin, and combinations thereof.

The composition may further comprise a vitamin selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), choline and combinations thereof.

The composition may further comprise a mineral selected from the group consisting of boron, calcium, chromium, copper, iodine, iron, magnesium, manganese, molybdenum, nickel, phosphorus, potassium, selenium, silicon, tin, vanadium, zinc, and combinations thereof.

The composition may further comprise a plant extract selected from the group consisting of the Malvaceae family, such as Abehnoschus moschatus Medik, or from the Asteraceae family, such as Achiliea santolina L., or Achyrocline satureioides (Lam.) DC, or Artemisia dracunculus L. (dragon herb) or Cichorium inlybus L., or Eclipta alba (L) Hassk, or from the Lamiaccae family such as Ajuga iva L. Schreberr (Medit), or Ocimum sanctum Linn. (Tulasi), or Origanum vulgare L. or from the Annonaceae family such as Annona squamosa L., or from the Gentianeacea family such as Anthocleista djalonensis A. Chev (cabbage tree), or Anthocleista Schweinfiirthii, or Anthocleista vogelii Planch, Enicostemma littorale Blume or Genitalia olivieri L., or from the Oxalidaceae family such as Averrhoa bilimbi L, or Biophytum sensitivum (L) DC, or from the Leguminosae family such as Bauhinia candicans Benth, or from the Bixaceae family such as Bixa orellana L., or from the Nyciaginaceae family such as Boerhaavia diffusa L., or from the Brassicaceae family such as Brassica nigra (L) Koch, or Eruka sativa, or Lepidium sativum L., or from the Caesalpiniaceae family such as Capparis spinosa L. or Cassia auriculata L. or Tamarindus indica 6L., or from the Capparidaceae family such as Capparis spinosa L., or from the Apiaceae family such as Carum carvi L., or from the family Rutaceae such as Clausena anisata (Wild) Benth., or from the family Palmae such as Cocos nucifera Linn. (Coconut palm), or from the Cucurbitaceae family such as Cogniauxia podoleana, or Ibervillea sonorae S., or from the family Conimelinaceae such as Commelina communis L., or from the family Zingiberaceae such as Curcuma longa L., or from the family Poaceae such as Cynodon dactylon Pers. (Bermuda grass), or from the family Ginkgoaceae such as Ginkgo biloba L., or from the family Papilionaceae such as Glycyrrhiza uralensis Fish., or from the family Asclepiadaceae, such as Gongronema latifolium Benth., or Gymnema montanum Hook, or from the family Sterculiaceae such as Helicteres isora L., As., or from the Rubiaceae family such as Hintonia standleyana, or from the Gramineae family such as Hordeum vulgare L. (Barley), or from the Convolvulaceae family such as Ipomoea aquatic Forsk., or Ipomea batata Linn (Sweet potato), or from the Loranthaceae family such as Loranthus micranthus Linn, or from the Moraceae family such as Morus indica. L., or from the Musaceae family such as Musa sapientum Kuntz (Banana), or from the Euphorbiaceae family such as Phyllanthus amarus Schum. Thonn, or Phyllanthus niruri L., or Phyllanthus sellowianus Mull. Arg., or from the Piperacea family such as Piper longum, or from the Myrtaceae family such as Psidium guajava L., or Syzygium alternifolium (Wt) Walp, or from the Lythraceae family such as Punica granatum L. (pomegranate), or from the Papilionaceae family such as Retama raetam (RR) (Forssk) Webb., or from the Adoxaceae family such as Sambucus nigra L., or from the Apocynaceae family such as Sanguis draxonis, or from the Anacardiaceae family such as Sclerocarya birea (A. Rich), or from the Scrophariaceae family such as Scoparia dulcis L., or from the Caryophyllaceae family such as Spergularia purpurea, or from the Chenopodiaceae family such as Suaeda fruticosa (SF) Euras, or from the Combretaceae family such as Terminalia chebula Retz., or Terminalia bellirica (Gaertn), or from the Menispermaceae family such as Tinospora cordifolia Miers., or from the Urticaceae family such as Urtica pilulifera L., or from the Astereaceae family such as Vernonia amygdalina Del., or from the Solanaceae family such as Withania soimifera (L) Dunal, or from the Zygophyllaceae family such as Zygophyllum gaetulum Emb and Maire and combinations thereof.

In a fourth aspect, the present invention provides an aqueous composition comprising: 2.40 mg/mL papain; 2.60 mg/mL dry Pinus pinaster stem bark extract; 1.75 mg/mL dry Aloe vera inner leaf juice; 100.00 mg/mL honey; 2.00 mg/mL glacial acetic acid; 3.00 mg/mL sodium chloride; 400 μg/mL potassium sorbate; 400 μg/mL sodium benzoate; 10.00 mg/mL wildberry flavour UA71225; and 800 μg/mL anthocyanin extract.

In a fifth aspect, the present invention provides an aqueous composition comprising papain, Pinus pinaster stem bark extract, and Aloe vera inner leaf juice in a ratio of papain:dry Pinus pinaster stem bark extract:dry Aloe vera inner leaf juice of 2.40:2.60: 1.75 by weight.

In one embodiment, there is provided an aqueous composition comprising papain, Pinus pinaster stem bark extract, Aloe vera inner leaf juice, and honey in a ratio of papain:dry Pinus pinaster stern bark extract:dry Aloe vera inner leaf juice:honey of 2.40:2.60: 1.75:100 by weight.

In another embodiment, there is provided an aqueous composition comprising papain, Pinus pinaster stem bark extract, Aloe vera inner leaf juice, and glacial acetic acid in a ratio of papain:dry Pinus pinaster stem bark extract:dry Aloe vera inner leaf juice:glacial acetic acid of 2.40: 2.60:1.75:2.00 by weight.

In another embodiment, there is provided an aqueous composition comprising papain, Pinus pinaster stem bark extract, Aloe vera inner leaf juice, honey, and glacial acetic acid in a ratio of papain:dry Pinus pinaster stem bark extract:dry Aloe vera inner leaf juice:honey : glacial acetic acid of 2.40:2.60:1.75:100:2.00 by weight.

In yet another embodiment, there is provided an aqueous composition comprising papain, Pinus pinaster stern bark extract, Aloe vera inner leaf juice, honey, glacial acetic acid, and sodium chloride in a ratio of papain:dry Pinus pinaster stem bark extract:dry Aloe vera inner leaf juice:honey:glacial acetic acid:sodium chloride of 2.40:2.60: 1.75:100:2.00:3.00 by weight.

In a further embodiment, there is provided an aqueous composition comprising papain, dry Pinus pinaster stem bark extract, dry Aloe vera inner leaf juice, honey, glacial acetic acid, sodium chloride, potassium sorbate, sodium benzoate, wildberry flavour UA71225 and anthocyanin extract in a ratio of papain:dry Pinus pinaster stem bark extract:dry Aloe vera inner leaf juice:honey:glacial acetic acid:sodium chloride:potassium sorbate:sodium benzoate:wildberry flavour UA71225:anthocyanin extract of 2.40:2.60:1.75:100:2.00:3.00:0.40:0.40:10.00:0.80 by weight.

In a sixth aspect, the present invention provides a method of treating or preventing elevated blood glucose, comprising administering to a subject in need thereof an effective amount of a composition according to any one of the first to fifth aspects above.

In a seventh aspect, the present invention provides a method of preventing or delaying the onset of type 2 diabetes mellitus (T2DM), comprising administering to a subject in need thereof an effective amount of a composition according to any one of the first to fifth aspects above.

In an eighth aspect, the present invention provides a method of treating or preventing pre-diabetes, comprising administering to a subject in need thereof an effective amount of a composition according to any one of the first to fifth aspects above.

In a ninth aspect, the present invention provides a method of reducing inflammation, comprising administering to a subject in need thereof an effective amount of a composition according to any one of the first to fifth aspects above.

In a tenth aspect, the present invention provides a method of decreasing cytokine-induced inflammation comprising administering to a subject in need thereof an effective amount of a composition according to any one of the first to fifth aspects above.

In an eleventh aspect, the present invention provides a method of decreasing an oxidative stress response comprising administering to a subject in need thereof an effective amount of a composition according to any one of the first to fifth aspects above.

In a twelfth aspect, the present invention provides a method of treating or preventing a disease characterised by elevated levels of cytokine-induced inflammation, comprising administering to a subject in need thereof an effective amount of a composition according to any one of the first to fifth aspects above.

In a thirteenth aspect, the present invention provides a method of treating or preventing a disease characterised by elevated levels of oxidative stress, comprising administering to a subject in need thereof an effective amount of a composition according to any one of the first to fifth aspects above.

In one embodiment of the twelfth or thirteenth aspects above, the disease is atherosclerosis, liver inflammation associated with type 2 diabetes and obesity, endometriosis, or osteoarthritis.

In a fourteenth aspect, the present invention provides a method of reducing one or more side effects of a diabetes medication selected from the group consisting of metformin, sulfonylureas, meglitinides, thiazolidinediones, DPP-4 inhibitors, GLP-1 receptor agonists, SGLT2 inhibitors, and insulin therapy, the method comprising administering to a subject in need thereof an effective amount of a composition according to any one of the first to fifth aspects above. The side effect may be abdominal or stomach discomfort, decreased appetite, or diarrhoea.

In a fifteenth aspect, the present invention provides a method of treating or preventing a dysfunction associated with excess blood glucose, comprising administering to a subject in need thereof an effective amount of a composition according to any one of the first to fifth aspects above. The dysfunction may be selected from the group consisting of: increased thirst and hunger, frequent urination, fatigue/lethargy, blurry vision, sleepiness, and muscle weakness.

In one embodiment of the sixth to fifteenth aspects above, the composition is administered in combination with metformin or another blood glucose lowering medication.

In a sixteenth aspect, the present invention provides a method of improving organ function comprising administering to a subject in need thereof an effective amount of a composition according to any one of the first to fifth aspects above.

In a seventeenth aspect, the present invention provides a method of lowering blood cholesterol, comprising administering to a subject in need thereof an effective amount of a composition according to any one of the first to fifth aspects above. The blood cholesterol may be selected from the group consisting of: low density lipoproteins (LDL), high density lipoproteins (HDL), trigylcerides and total cholesterol. In one embodiment, the present invention provides a method of lowering low density lipoprotein (LDL) blood cholesterol levels, comprising administering to a subject in need thereof an effective amount of a composition according to any one of the first to fifth aspects above. In another embodiment, the present invention provides a method of increasing high density lipoprotein (HDL) blood cholesterol levels, comprising administering to a subject in need thereof an effective amount of a composition according to any one of the first to fifth aspects above. In yet another embodiment, the present invention provides a method of decreasing the ratio of Total Cholesterol to HDL, comprising administering to a subject in need thereof an effective amount of a composition according to any one of the first to fifth aspects above. In yet another embodiment, the present invention provides a method of decreasing blood triglyceride levels, comprising administering to a subject in need thereof an effective amount of a composition according to any one of the first to fifth aspects above.

The effective amount of the composition may correspond to: a daily adult human dosage of from about 60 mg to about 1500 mg Pinus pinaster stem bark extract; a daily adult human dosage of from about 30 mg to about 1200 mg papain; and a daily adult human dosage of from about 15 mg to about 600 mg Aloe vera extract. For example, the effective amount of the composition may correspond to: a daily adult human dosage of about 260 mg Pinus pinaster stem bark extract; a daily adult human dosage of about 240 mg papain; and a daily adult human dosage of about 175 mg Aloe vera extract. The daily adult human dosage of Pinus pinaster stem bark extract, papain and Aloe vera extract may be provided to the subject in a single dose, or the daily adult human dosage of Pinus pinaster stem bark extract, papain and Aloe vera extract may be provided to the subject in multiple doses.

In one embodiment of the sixth to seventeenth aspects above, the subject is selected from the group consisting of the elderly, those with a medical condition of pre-diabetes or at risk of developing pre-diabetes, and combinations thereof.

In an eighteenth aspect, the present invention provides a method of improving immune function, comprising administering to a subject in need thereof an effective amount of a composition according to any one of the first to fifth aspects above. In one embodiment, improved immune function is characterised by an increased white blood cell count, e.g., increased levels of neutrophils.

In a nineteenth aspect, the present invention provides a method of treating an autoimmune disease, comprising administering to a subject in need thereof an effective amount of a composition according to any one of the first to fifth aspects above. The autoimmune disease may be selected from the group consisting of: lupus, arthritis, psoriasis, and type I diabetes. In one embodiment, the present invention provides a method of treating lupus, comprising administering to a subject in need thereof an effective amount of a composition according to any one of the first to fifth aspects above. The treatment of lupus may reduce the number of lesions, e.g., sub cutaneous lesions, in or on the subject. In another embodiment, the present invention provides a method of treating arthritis, comprising administering to a subject in need thereof an effective amount of a composition according to any one of the first to fifth aspects above. In one embodiment, the arthritis is rheumatoid arthritis. In a further embodiment, the present invention provides a method of treating psoriasis, comprising administering to a subject in need thereof an effective amount of a composition according to any one of the first to fifth aspects above. In yet a further embodiment, the present invention provides a method of treating type I diabetes, comprising administering to a subject in need thereof an effective amount of a composition according to any one of the first to fifth aspects above.

In a twentieth aspect, the present invention provides a method of treating emphysema, comprising administering to a subject in need thereof an effective amount of a composition according to any one of the first to fifth aspects above.

In a twenty first aspect, the present invention provides a method of increasing general wellbeing, comprising administering to a subject in need thereof an effective amount of a composition according to any one of the first to fifth aspects above. In one embodiment, a measure of general wellbeing includes one or more of: increased energy levels, improved vitality, better sleep, improved breathing ability, improved levels of physical activity, improved mental functioning, and/or improved physical functioning.

In the method of any one of the sixth to twenty first aspects above, the composition may be administered to an adult human subject at a dosage of 30 to 100 mL per day, wherein each mL of the composition comprises: Pinus pinaster (Maritime Pine) stem bark concentrated extract equivalent to 1.56 g dry Pinus pinaster stem bark standardised to contain 1.82 mg procyanidins, 2.4 mg papain, 3 mg sodium chloride, and Aloe vera dried inner leaf juice equivalent to 350 mg fresh juice. In an embodiment, each mL of the composition comprises: 2.40 mg papain; 2.60 mg dry Pinus pinaster stem bark extract; 1.75 mg dry Aloe vera inner leaf juice; 100.00 mg honey; 2.00 mg glacial acetic acid; 3.00 mg sodium chloride; 400 μg potassium sorbate; 400 μg sodium benzoate; 10.00 mg wildberry flavour UA71225; and 800 μg anthocyanin extract, or equivalents thereof. The composition may be administered at a total daily dosage of about 30 to about 100 mL in two separate doses. For example, the composition may be administered in two separate doses of about 15 to about 50 mL, one dose after a morning meal and one dose after an evening meal.

In a twenty second aspect, the present invention provides use of a composition according to any one of the first to fifth aspects above for the manufacture of a medicament for: treating or preventing elevated blood glucose; or preventing or delaying the onset of type 2 diabetes mellitus (T2DM); or treating or preventing pre-diabetes; or reducing inflammation; or decreasing cytokine-induced inflammation; or decreasing an oxidative stress response; or treating or preventing a disease characterised by elevated levels of cytokine-induced inflammation; or treating or preventing a disease characterised by elevated levels of oxidative stress; or reducing one or more side effects of a diabetes medication selected from the group consisting of metformin, sulfonylureas, meglitinides, thiazolidinediones, DPP-4 inhibitors, GLP-1 receptor agonists, SGLT2 inhibitors, and insulin therapy; or treating or preventing a dysfunction associated with excess blood glucose; or improving organ function; or lowering blood cholesterol in a subject.

In a twenty third aspect, the present invention provides use of a composition according to any one of the first to fifth aspects above for the manufacture of a medicament for: improving immune function; treating an autoimmune disease, e.g., lupus, arthritis, psoriasis, or type I diabetes; treating emphysema; or increasing general wellbeing, e.g., increasing energy levels, improving vitality, improving sleep, improving breathing ability, improving levels of physical activity, improving mental functioning, and/or improving physical functioning in a subject.

In one embodiment, the present invention provides use of a composition according to any one of the first to fifth aspects above for the manufacture of a medicament for treating or preventing elevated blood glucose in a subject.

In one embodiment, the present invention provides use of a composition according to any one of the first to fifth aspects above for the manufacture of a medicament for preventing or delaying the onset of type 2 diabetes mellitus (T2DM) in a subject.

In one embodiment, the present invention provides use of a composition according to any one of the first to fifth aspects above for the manufacture of a medicament for treating or preventing pre-diabetes in a subject.

In one embodiment, the present invention provides use of a composition according to any one of the first to fifth aspects above for the manufacture of a medicament for reducing inflammation in a subject.

In one embodiment, the present invention provides use of a composition according to any one of the first to fifth aspects above for the manufacture of a medicament for decreasing cytokine-induced inflammation in a subject.

In one embodiment, the present invention provides use of a composition according to any one of the first to fifth aspects above for the manufacture of a medicament for decreasing an oxidative stress response in a subject.

In one embodiment, the present invention provides use of a composition according to any one of the first to fifth aspects above for the manufacture of a medicament for treating or preventing a disease characterised by elevated levels of cytokine-induced inflammation in a subject. The disease may be atherosclerosis, liver inflammation associated with type 2 diabetes and obesity, endometriosis, or osteoarthritis.

In one embodiment, the present invention provides use of a composition according to any one of the first to fifth aspects above for the manufacture of a medicament for treating or preventing a disease characterised by elevated levels of oxidative stress in a subject. The disease may be atherosclerosis, liver inflammation associated with type 2 diabetes and obesity, endometriosis, or osteoarthritis.

In one embodiment, the present invention provides use of a composition according to any one of the first to fifth aspects above for the manufacture of a medicament for reducing one or more side effects of a diabetes medication selected from the group consisting of metformin, sulfonylureas, meglitinides, thiazolidinediones, DPP-4 inhibitors, GLP-1 receptor agonists, SGLT2 inhibitors, and insulin therapy in a subject. The side effect may be abdominal or stomach discomfort, decreased appetite, or diarrhoea.

In one embodiment, the present invention provides use of a composition according to any one of the first to fifth aspects above for the manufacture of a medicament for treating or preventing a dysfunction associated with excess blood glucose in a subject. The dysfunction may be selected from the group consisting of: increased thirst and hunger, frequent urination, fatigue/lethargy, blurry vision, sleepiness, and muscle weakness.

In one embodiment, the present invention provides use of a composition according to any one of the first to fifth aspects above for the manufacture of a medicament for improving organ function in a subject.

In one embodiment, the present invention provides use of a composition according to any one of the first to fifth aspects above for the manufacture of a medicament for lowering blood cholesterol in a subject.

In one embodiment of the twenty second aspect above, the subject may be taking metformin or another blood glucose lowering medication, and/or the subject may be selected from the group consisting of the elderly, those with a medical condition of pre-diabetes or at risk of developing pre-diabetes, and combinations thereof.

In one embodiment, the present invention provides use of a composition according to any one of the first to fifth aspects above for the manufacture of a medicament for improving immune function in a subject. In another embodiment, the present invention provides use of a composition according to any one of the first to fifth aspect above for treating an autoimmune disease, e.g., lupus, arthritis, psoriasis, or type I diabetes in a subject. In a further embodiment, the present invention provides use of a composition according to any one of the first to fifth aspects above for the manufacture of a medicament for treating emphysema in a subject. In yet a further embodiment, the present invention provides use of a composition according to any one of the first to fifth aspects above for the manufacture of a medicament for increasing general wellbeing, e.g., increasing energy levels, improving vitality, improving sleep, improving breathing ability, improving levels of physical activity, improving mental functioning, and/or improving physical functioning in a subject.

### BRIEF DESCRIPTION OF THE DRAWINGS

Embodiments of the present invention will now be described, by way of example only with reference to the accompanying figures wherein:

FIG. 1 shows that a composition according to Example 1 decreases inflammatory markers (a) TNF-alpha-induced VCAM-1, (b) TNF-alpha-induced ICAM-1 and (c) TNF-alpha-induced ELAM in human coronary artery endothelial cells. HCAECs were exposed to the indicated amounts (μL/mL) of the composition of Example 1 for 3 hours prior to exposure to TNF-alpha.

FIG. 2 shows that a composition according to Example 1 decreases protein levels of (a) TNF-alpha-induced VCAM-1 and (b) TNF-alpha-induced ICAM-1 in human coronary artery endothelial cells. HCAECs were exposed to the indicated amounts (μL/mL) of the composition of Example 1 for 3 hours prior to exposure to TNF-alpha.

FIG. 3 shows that a composition according to Example 1 decreases activation of the key mediator of inflammation, NFkB. HCAECs were exposed to the indicated amounts (μL/mL) of the composition of Example 1 prior to exposure to TNF-alpha.

FIG. 4 shows that a composition according to Example 1 decreases cytokine-induced ROS levels in TNF-alpha-induced HCAECs. HCAECs were exposed to the indicated amounts (μL/mL) of the composition of Example 1 for 3 hours prior to exposure to TNF-alpha.

FIG. 5 shows that a composition according to Example 1 decreases (a) NOX-4 and increases (b) SOD-1 levels in TNF-alpha-induced HCAECs. HCAECs were exposed to the indicated amounts (μL/mL) of the composition of Example 1 prior to exposure to TNF-alpha.

FIG. 6 shows that a composition according to Example 1 ingested by athletes pre-training decreases CRP levels in their blood post-training (dark grey=runners, light grey=cyclists).

FIG. 7 shows that a composition according to Example 1 blocks VCAM-1 levels from rising in response to the pro-inflammatory cytokine, TNF-α. HCAECs were exposed to concentrations equivalent to a 50 mL, 25 mL, 12.5 mL and 6.25 mL dose of the composition of Example 1 prior to exposure to TNF-alpha.

FIG. 8 shows that human coronary artery endothelial cells exposed to an inflammatory stimulus, TNF-α, after pre-treatment (24 h) with plasma obtained from individuals 3 h after ingestion of a composition of Example 1 demonstrated a reduced level of inflammation response (measured by VCAM-1 levels) compared to cells pretreated with plasma from the same individuals pre-ingestion of the composition.

### DEFINITIONS

As used in this application, the singular form “a”, “an” and “the” include plural references unless the context clearly dictates otherwise. For example, the phrase ‘a composition’ also includes a plurality of compositions.

As used herein, the term “comprising” means “including.” Variations of the word “comprising”, such as “comprise” and “comprises,” have correspondingly varied meanings. Thus, for example, a composition “comprising” Pinus pinaster stem bark extract, papain and Aloe vera extract may consist exclusively of Pinus pinaster stem bark extract, papain and Aloe vera extract, or it may include one or more additional components (e.g., acid, honey, preservatives, etc.).

It will be understood that use the term “about” herein in reference to a recited numerical value includes the recited numerical value and numerical values within plus or minus ten percent of the recited value.

As used herein, the term “subject” is understood to include an animal, especially a mammal, and more especially a human, that is receiving or intended to receive treatment or has or is at risk of having a medical condition that can benefit from the composition as described herein. For example, the animal may include, but is not limited to, mammals such as rodents, aquatic mammals, domestic animals such as dogs and cats, farm animals such as sheep, pigs, cows and horses, and humans. It is also contemplated that the term ‘subject’ applies to any animals that are capable of the effect exhibited or intended to be exhibited by the compositions as described herein.

It will be understood that use of the term “between” herein when referring to a range of numerical values encompasses the numerical values at each endpoint of the range. For example, a pH of between about 3.2 and about 4.2 is inclusive of a pH of 3.2 and a pH of 4.2.

As used herein, the terms “aqueous” and “aqueous composition” will be understood as referring to a composition formulated in or with water, and in particular, purified water. In some embodiments, the aqueous compositions will be predominantly water, for example, up to 99% water by weight or by volume. In other embodiments, the aqueous compositions will comprise at least some water, for example, more than 1% water by weight or by volume.

### DETAILED DESCRIPTION

The following detailed description conveys the present invention, including exemplary embodiments thereof, in sufficient detail to enable those of ordinary skill in the art to practice the present invention. Features or limitations of the various embodiments described do not necessarily limit other embodiments of the present invention or the present invention as a whole. Hence, the following detailed description does not limit the scope of the present invention, which is defined only by the claims.

The present disclosure relates generally to a composition comprising Pinus pinaster stem bark extract in combination with Aloe vera extract (alternatively known as A. barbadensis Mill., Aloe indica Royle, Aloe perfoliata L. var. vera and A. vulgaris Lam) and papain and methods and uses thereof for the therapeutic and/or prophylactic treatment, amelioration and/or regulation of disease states or pathological conditions that are associated with excess blood glucose (hyperglycemia), for improving organ function, and for reducing inflammation. The compositions and methods may, for example, be suitable for treating and/or preventing a dysfunction associated with excess blood glucose, which may include disturbances in everyday physiological functions such as increased thirst and hunger, frequent urination, fatigue/lethargy, blurry vision, sleepiness, and/or muscle weakness. Such associated conditions may be compounded by pharmaceutical administrations (e.g., metformin), which may cause or exacerbate abdominal or stomach discomfort, decreased appetite, diarrhoea, and a general feeling of discomfort.

### Composition

Pinus pinaster Stem Bark Extract

The composition according to the present invention comprises Pinus pinaster stem bark extract. This Pinus pinaster stem bark extract is made from the bark of a European coastal pine tree called the Landes or maritime pine, whose scientific name is Pinus maritima or Pinus pinaster. Without being bound by theory, the pine bark extract may have antioxidant properties that make it suitable for a wide range of healing and preventative purposes.

The Pinus pinaster stem bark extract may be derived from any suitable source, for example, extracted from the bark of the maritime pine tree using methods known in the art, or it may be obtained commercially, e.g., as the product Pycnogenol® supplied by Horphag Research USA Inc. or as Pinus pinaster L supplied by Pharmactive Biotech Products SL. Preferably, the Pinus pinaster stem bark extract is Pycnogenol® supplied by Horphag Research USA Inc. The Pinus pinaster stern bark extract may be provided in the composition according to the invention in solid form, e.g., as a dry powder, or a dry powder compacted into a tablet, or dry powder in a capsule, or in a liquid form, e.g., dry powder dissolved or suspended in a solution, suspension or emulsion. Where the composition comprising Pinus pinaster stem bark extract is provided as an aqueous composition, dry powdered Pinus pinaster stem bark extract may be dissolved in the water. In the context of this specification, the term ‘extract’ is to be given its broadest construction and is understood to refer to a plant and its components or preparation, or component of a plant or herb or preparation derived therefrom, which, when used in the composition according to the invention in an effective amount, promotes a health benefit in the subject such as regulation of high blood glucose levels.

The Pinus pinaster stern bark extract may be present in the composition in any suitable amount, e.g., at a therapeutically effective amount. A therapeutically effective amount of Pinus pinaster stem bark extract may correspond to a daily adult human dosage of from about 60 mg to about 1500 mg Pinus pinaster stem bark extract. In one embodiment, the Pinus pinaster stem bark extract is present in the composition in a therapeutically effective amount of corresponding to a daily adult human dosage of about 260 mg.

Where the composition is formulated as liquid, e.g., an aqueous liquid, Pinus pinaster stem bark extract may be present in the composition in a concentration ranging from about 2.0 mg/mL to about 10 mg/mL. For example, Pinus pinaster stem bark extract may be present in the composition in a concentration ranging from about 2.0 mg/mL to about 3.0 mg/mL, or from about 2.0 mg/mL to about 5.0 mg/mL, or from about 4.0 mg/mL to about 8.0 mg/mL, or from about 6.0 mg/mL to about 10.0 mg/mL, e.g., at a concentration of 2.0 mg/mL, 2.2 mg/mL, 2.4 mg/mL, 2.6 mg/mL, 2.8 mg/mL, 3.0 mg/mL, 3.2 mg/mL, 3.4 mg/mL, 3.6 mg/mL, 3.8 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, or 10 mg/mL, where the amount of Pinus pinaster stem bark extract is expressed as the amount of dry Pinus pinaster stem bark extract per mL of the composition. It is contemplated herein that the amount of dry Pinus pinaster stem bark extract per mL includes Pinus pinaster stem bark extract added to the liquid composition in dry form, but also covers addition of Pinus pinaster stem bark extract in other forms (e.g., in a liquid concentrate) in different amounts, provided that the overall concentration of Pinus pinaster stem bark extract after addition is equivalent to the concentration of dry Pinus pinaster stem bark extract per mL of the composition.

The amounts of “dry Pinus pinaster stem bark extract” referred to herein are calculated on the basis of the exemplified embodiments of the invention, for example, the composition in Table 1, in which the dry Pinus pinaster stem bark extract added to the composition comprises approximately 65-75% procyanidins. In one embodiment, the 2.60 mg dry Pinus pinaster stem bark extract per mL in Table 1, equivalent to 1.56 g dry MIMS pinaster stem bark, is standardised to contain about 1.82 mg procyanidins. It will be understood that alternative sources or forms of Pinus pinaster stem bark extract may be available, in which case the amount of Pinus pinaster stem bark extract used in the composition of the invention may be adjusted so as to provide an equivalent amount of procyanidins rather than a necessarily equivalent amount of Pinus pinaster stem bark extract.

In one embodiment, the Pinus pinaster stem bark extract is present in the aqueous composition in a concentration of 2.6 mg/mL.

### Papain

The composition according to the present invention comprises papain. Papain is a cysteine protease enzyme extracted from the papaya plant (Carica papaya).

The papain may be derived from any suitable source, for example, the leaves, latex, roots, and/or fruits of the papaya plant using methods known in the art, or it may be obtained commercially, e.g., as the product Papaina (USP) as supplied by Biocon Limited, Bangalore, India. It may be added to the composition according to the invention in solid form, e.g., as a dry powder, or in any other suitable form, e.g., suspended or dissolved in an aqueous buffer solution, or as a concentrated liquid. Where the composition comprising papain is provided as an aqueous composition, pure, powdered, dry papain may be dissolved in the water.

The papain may be present in the composition in any suitable amount, e.g., at a therapeutically effective amount. A therapeutically effective amount of papain may correspond to a daily adult human dosage of from about 30 mg to about 1200 mg papain. In one embodiment, the papain is present in the composition in a therapeutically effective amount corresponding to a daily adult human dosage of about 240 mg.

Where the composition is formulated as liquid, e.g., an aqueous liquid, papain may be present in the composition in a concentration ranging from about 1.0 mg/mL to about 8 mg/mL. For example, papain may be present in the composition in a concentration ranging from about 1.0 mg/mL to about 3.0 mg/mL, or from about 2.0 mg/mL to about 5.0 mg/mL, or from about 4.0 mg/mL to about 6.0 mg/mL, or from about 5.0 mg/mL to about 8.0 mg/mL, e.g., at a concentration of 1.0 mg/mL, 1.2 mg/mL, 1.4 mg/mL, 1.6 mg/mL, 1.8 mg/mL, 2.0 mg/mL, 2.2 mg/mL, 2.4 mg/mL, 2.6 mg/mL, 2.8 mg/mL, 3.0 mg/mL, 3.2 mg/mL, 3.4 mg/mL, 3.6 mg/mL, 3.8 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, or 8.0 mg/mL, where the amount of papain is expressed as the amount of dry papain per mL of the composition. It is contemplated herein that the amount of dry papain per mL includes papain added to the liquid composition in dry form, but also covers addition of papain in other forms (e.g., in a liquid concentrate) in different amounts, provided that the overall concentration of papain after addition is equivalent to the concentration of dry papain per mL of the composition.

In one embodiment, the papain is present in the aqueous composition in a concentration of 2.4 mg/mL.

Aloe vera

The composition according to the present invention comprises Aloe vera extract. Aloe vera is alternatively known as A. barbadensis Mill., Aloe indica Royle, Aloe perfoliata L. var. vera and A. vulgaris Lam. The Aloe vera extract may be extracted from any suitable part of the Aloe vera plant, for example, the Aloe vera extract may be Aloe vera leaf extract, e.g., made by crushing the whole Aloe vera leaf, or may be Aloe vera gel extract, or may be Aloe vera latext extract. Preferably, the Aloe vera extract is dry Aloe vera inner leaf juice extract.

The Aloe vera extract may be derived from any suitable source, for example, the leaves, latex, or gel of the Aloe vera plant using methods known in the art or it may be obtained commercially, e.g., as the product Aloe barbadensis Miller, spray dried supplied by Capitol Ingredients. It may be utilised in the composition according to the invention in solid form, e.g., as a dry inner leaf juice powder, or in any other suitable form, e.g., as a liquid, gel, or paste. Thus, as used herein, Aloe vera extract may refer to dry Aloe vera inner leaf juice extract. Where the composition comprising Aloe vera extract is provided as an aqueous composition, powdered dry Aloe vera inner leaf juice extract may be dissolved in the water.

The Aloe vera extract may be present in the composition in any suitable amount, e.g., in a therapeutically effective amount. A therapeutically effective amount of Aloe vera extract may correspond to a daily adult human dosage of from about 15 mg to about 600 mg Aloe vera extract. In one embodiment, the Aloe vera extract, e.g., Aloe vera inner leaf juice extract, is present in the composition in a therapeutically effective amount corresponding to a daily adult human dosage of about 175 mg.

Where the composition is formulated as liquid, e.g., an aqueous liquid, Aloe vera inner leaf juice extract may be present in the composition in a concentration ranging from about 0.5 mg/mL to about 4 mg/mL. For example, Aloe vera inner leaf juice extract may be present in the composition in a concentration ranging from about 0.5 mg/mL to about 2.0 mg/mL, or from about 1.5 mg/mL to about 3.5 mg/mL, or from about 2.0 mg/mL to about 4.0 mg/mL, e.g., at a concentration of 0.5 mg/mL, 0.7 mg/mL, 0.9 mg/mL, 1.0 mg/mL, 1.2 mg/mL, 1.4 mg/mL, 1.5 mg/mL, 1.6 mg/mL, 1.7 mg/mL, 1.8 mg/mL, 1.9 mg/mL, 2.0 mg/mL, 2.2 mg/mL, 2.4 mg/mL, 2.6 mg/mL, 2.8 mg/mL, 3.0 mg/mL, 3.2 mg/mL, 3.4 mg/mL, 3.6 mg/mL, 3.8 mg/mL, or 4.0 mg/mL, where the amount of Aloe vera inner leaf juice extract is expressed as the amount of spray dried Aloe vera inner leaf juice extract per mL of the composition. It is contemplated herein that the amount of spray dried Aloe vera inner leaf juice extract per mL includes spray dried Aloe vera inner leaf juice extract added to the liquid composition in dry form, but also covers addition of Aloe vera inner leaf juice extract in other forms (e.g., in a liquid or gel concentrate) in different amounts, provided that the overall concentration of Aloe vera inner leaf juice extract after addition is equivalent to the concentration of spray dried Aloe vera inner leaf juice extract per mL of the composition. In this regard, the amounts of “spray dried Aloe vera inner leaf juice extract” referred to herein are calculated on the basis of the exemplified embodiments of the invention, for example, the composition in Table 1, in which the spray dried Aloe vera inner leaf juice extract added to the composition comprises approximately 40-50% maltodextrin and wherein the ratio of fresh plant to spray dried material is about 180-210:1. It will be understood that alternative methods for the preparation of Aloe vera extract may be available, in which case the amount of Aloe vera extract used in the composition of the invention may be adjusted to account for differences in the concentration of actual Aloe vera sourced material and or additives present.

In one embodiment, the Aloe vera inner leaf juice extract is present in the aqueous composition in a concentration of 1.75 mg/mL.

The composition of the present disclosure including Pinus pinaster stem bark extract, papain and Aloe vera extract may be administered in combination with other compounds to assist in the regulation of blood glucose levels. This may permit tailoring of the taste profile of the compositions to improve compliance and thus efficacy and/or to deliver additional or complementary health benefits.

### Honey

Accordingly, the composition according to the present invention may comprise honey. Any suitable honey or mixture of honeys may be used.

The honey may be derived from any suitable source from any suitable floral species, e.g., it may be obtained commercially as British Pharmacopoeia (BP) grade honey, for example, as supplied by Capilano Honey Limited, Australia. The honey may be added to the composition according to the invention as a pure honey in the form of a concentrated, viscous mixture. It may be heated prior to addition to lower the viscosity and increase ease of dispersion or solubilisation in the composition.

The honey may be present in the composition in any suitable amount. For example, pure honey may be present in the composition at a daily adult human dosage of from about 1.5 g to about 30 g/day, e.g., at a daily human dosage of from about 1.5 g to about 5 g, or from about 5 g to about 10 g, or from about 7 g to about 12 g, or from about 10 g to about 20 g, or from about 15 g to about 30 g, or from about 20 g to about 25 g, or at a daily human dosage of about 1.5 g, 2 g, 5 g, 7.5 g, 10 g, 12.5 g, 15 g, 17.5 g, 20 g, 22.5 g, 25 g, 27.5 g, or 30 g. In one embodiment, the honey is present in the composition in an amount corresponding to a daily adult human dosage of about 10 g.

Where the composition is formulated as liquid, e.g., an aqueous liquid, pure honey may be present in a concentration ranging from about 50 mg/mL to about 200 mg/mL. For example, pure honey may be present in the composition in a concentration ranging from about 50 mg/mL to about 100 mg/mL, or from about 75 mg/mL to about 150 mg/mL, or from about 100 mg/mL to about 200 mg/mL, e.g., at a concentration of 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 1900 mg/mL, or 200 mg/mL. In one embodiment, the honey is present in the aqueous composition in a concentration of 100 mg/mL.

### Acid

The composition according to the present invention may comprise an acid. Any suitable acid or mixture of acids may be used, for example, the acid may be an organic or an inorganic acid, or a mixture of any one or more organic and/or inorganic acids. The acid may be of any suitable concentration, e.g., may be concentrated (e.g., substantially anhydrous) or dilute. In one embodiment, the acid is a food acid, e.g., acetic acid. In a preferred embodiment, the acid is acetic acid. In another embodiment, the acid is glacial acetic acid. The acid may be added to assist the composition with entering the blood stream of a subject, which may in turn enable the composition or one or more of its component ingredients to reach parts of the subject's body that are, e g , inflamed. The acid may be added to the composition as vinegar.

The acid may be present in the composition in an amount sufficient to acidify the composition to a pH of between about 3.2 to about 4.2, for example, between about 3.2 and about 3.7, about 3.5 and about 4.0, about 3.7 and about 4.2, or about 4.0 and about 4.2, e.g., at a pH of about 3.2, about 3.3, about 3.4, about 3.5, about 3.6, about 3.7, about 3.8, about 3.9, about 4.0, about 4.1, or about 4.2.

Where the acid is glacial acetic acid, the acid may be present in the composition in any suitable amount. For example, the glacial acetic acid may be present in the composition at a daily adult human dosage of from about 30 mg to about 1200 mg/day, e.g., at a daily human dosage of from about 30 mg to about 100 mg, or from about 50 mg to about 250 mg, or from about 150 mg to about 250 mg, or from about 200 mg to about 500 mg, or from about 500 mg to about 750 mg, or from about 700 mg to about 1000 mg, or from about 900 mg to about 1200 mg, or at a daily human dosage of about 30 mg, 50 mg, 100 mg, 200 mg, 300 mg, 400 mg, 500 mg, 600 mg, 700 mg, 800 mg, 900 mg, 1000 mg, 1100 mg, or 1200 mg. In one embodiment, the glacial acetic acid is present in the composition in an amount corresponding to a daily adult human dosage of about 200 mg.

Where the composition is formulated as liquid, e.g., an aqueous liquid, the glacial acetic acid may be present in the composition in a concentration ranging from about 1 mg/mL to about 8 mg/mL. For example, the glacial acetic acid may be present in the composition in a concentration ranging from about 1.0 mg/mL to about 3.0 mg/mL, or from about 2.0 mg/mL to about 5.0 mg/mL, or from about 3.0 mg/mL to about 6.0 mg/mL, or from about 4.0 mg/mL to about 8.0 mg/mL, e.g., at a concentration of 1.0 mg/mL, 1.2 mg/mL, 1.4 mg/mL, 1.6 mg/mL, 1.8 mg/mL, 2.0 mg/mL, 2.2 mg/mL, 2.4 mg/mL, 2.6 mg/mL, 2.8 mg/mL, 3.0 mg/mL, 3.2 mg/mL, 3.4 mg/mL, 3.6 mg/mL, 3.8 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, or 8.0 mg/mL. In one embodiment, the glacial acetic acid is present in the aqueous composition in a concentration of 2 mg/mL.

The glacial acetic acid may be obtained commercially, e.g., as supplied by Bronson & Jacobs Pty Limited, Sydney, Australia.

### Synergism

The composition according to the present invention may comprise a synergistic mixture of active ingredients. The synergism may refer to the effect exerted by the composition in regulating blood sugar levels in a subject, or may refer to the effect exerted by the composition in treating or preventing pre-diabetes, diabetes, or any associated dysfunctions. For example, the composition comprising Pinus pinaster stem bark extract, papain and Aloe vera extract may act synergistically to regulate blood sugar levels or treat or prevent pre-diabetes, diabetes or any associated dysfunctions in a subject. The composition comprising Pinus pinaster stem bark extract, papain, Aloe vera extract and honey may additionally or alternatively act synergistically to regulate blood sugar levels or treat or prevent pre-diabetes, diabetes or any associated dysfunctions in a subject. The composition comprising Pinus pinaster stem bark extract, papain, Aloe vera extract, and acid, e.g., acetic acid, may additionally or alternatively act synergistically to regulate blood sugar levels or treat or prevent pre-diabetes, diabetes or any associated dysfunctions in a subject. The composition comprising Finns pinaster stem bark extract, papain, Aloe vera extract, honey and acid, e.g., acetic acid, may additionally or alternatively act synergistically to regulate blood sugar levels or treat or prevent pre-diabetes, diabetes or any associated dysfunctions in a subject.

### Additives

The composition of the invention may additionally include any suitable additives. For example, the additives may include a preservative, a flavouring agent, a salt, a pigment, or a mixture of any two or more of these.

Suitable preservatives are known in the art, for example benzoic acid, sodium benzoate, sodium or potassium sorbate, sorbic acid, hydroxybenzoate, etc. Accordingly, the composition disclosed herein may comprise one or more preservatives. The preservatives may be added to inhibit growth of bacterial or other harmful organisms in the composition during manufacture and storage. Suitable preservatives for use in the composition according to the present invention include sodium benzoate and potassium sorbate. Suitable preservatives may be added to the composition as disclosed herein in any suitable amount. For example, where the composition is formulated as liquid, e.g., an aqueous liquid, the preservatives may be present in a concentration ranging from about 500 μg/mL to about 1000 μg/mL, or from about 500 μg/mL to about 750 μg/mL, or from about 650 μg/mL to about 800 μg/mL, or from about 800 μg/mL to about 1000 pg/mL, e.g., about 500 μg/mL, about 600 μg/mL, about 700 μg/mL, about 800 μg/mL, about 900 pg/mL, or about 1000 μg/mL, or otherwise as may be determined by one skilled in the art. In one embodiment, sodium benzoate is present in the aqueous composition in a concentration of 400 μg/mL and potassium sorbate is present in the aqueous composition in a concentration of 400 μg/mL.

Suitable flavouring agents are also known in the art and are commercially available, e.g., suitable flavouring agents for use in the composition according to the present invention may include SM10483—Natural Strawberry Flavour(TIF-20550), SM00401—Natural Orange Oil Sweet Aust 48-7685, SM10458 Orange Passionfruit Flavour (UA-71735), or Wildberry flavour UA71225 as supplied by Ungerer Australia. Pty Limited. Accordingly, the composition disclosed herein may comprise one or more flavouring agents. The flavouring agents may improve the flavour and/or odour of the composition and result in increased compliance and therefore efficacy. Suitable flavouring agents may be added to the composition as disclosed herein in any suitable amount. For example, where the composition is formulated as liquid, e.g., an aqueous liquid, the flavouring agent may be present in a concentration ranging from about 5 mg/mL to 15 mg/mL, e.g., from about 5 mg/mL to about 7 mg/mL, from about 7 mg/mL to about 12 mg/mL, or from about 12 mg/mL to about 15 mg/mL, e.g., at about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, or otherwise as may be determined by one skilled in the art. In one embodiment, the flavouring agent—preferably Wildberry flavour UA71225—is present in the aqueous composition in a concentration of 10 mg/mL.

Suitable salts are known in the art, for example sodium and potassium chloride. Accordingly, the composition disclosed herein may comprise one or more salts. One suitable salt for use in the composition according to the present invention is sodium chloride. The salt may be added in any suitable amount. For example, where the composition is formulated as liquid, e.g., an aqueous liquid, the salt, preferably sodium chloride, may be present in a concentration ranging from about 1.0 mg/mL to about 6.0 mg/mL, e.g., in a range of from about 1.0 mg/mL to about 3.0 mg/mL, or from about 2.0 mg/mL to about 5.0 mg/mL, or from about 4.0 mg/mL to about 6.0 mg/mL, e.g., at a concentration of 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, or 6.0 mg/mL. In one embodiment, sodium chloride is present in the aqueous composition in a concentration of 3.00 mg/mL.

Suitable pigments are known in the art, for example carotenoids, chlorophylls and anthocyanins, etc. Accordingly, the composition disclosed herein may comprise one or more pigments. The pigments may be added to increase the visual appeal of the composition. Suitable pigments for use in the composition according to the present invention include anthocyanin extract. Suitable pigments may be added to the composition as disclosed herein in any suitable amount. For example, anthocyanin extract may be present in the composition in a concentration of between about 500 μg/mL and about 1000 μg/mL, or between about 500 μg/mL and about 750 μg/mL, or between about 650 μg/mL and about 800 μg/mL, or between about 800 μg/mL and about 1000 μg/mL, e.g., about 500 μg/mL, about 600 μg/mL, about 700 μg/mL, about 800 μg/mL, about 900 μg/mL, or about 1000 μg/mL, or otherwise as may be determined by one skilled in the art. In one embodiment, the pigment—preferably anthocyanin extract—is added to the aqueous composition at a concentration of 800 μg/mL.

The amounts of pigments, e.g., “anthocyanin extract” referred to herein are calculated on the basis of the exemplified embodiments of the invention, for example, the composition in Table 1, in which the anthocyanin extract added to the composition comprises approximately 25-35% maltodextrin. It will be understood that alternative sources or forms of anthocyanin extract may be available that contain more, less or no maltodextrin, in which case the amount of anthocyanin extract used in the composition of the invention may be adjusted so as to account for differences in the anthocyanin content of an anthocyanin extract used in the preparation of the inventive composition.

Other suitable additives, such as thickeners (e.g., maltodextrin), diluents, buffers, additional therapeutic agents, bioavailability enhancers, side-effect suppressing components, binders, etc. may also be used in the composition disclosed herein. These additives preferably do not negatively affect the efficacy or therapeutic benefits of the composition.

### Additional Components

In addition to the above additives, the composition according to the present invention may comprise one or more additional components.

For example, the composition may comprise an antioxidant such as ascorbic acid (vitamin C). Common additional antioxidants are vitamins A, E, and the mineral selenium or the group of antioxidants found in pine bark extract known as oligomeric proanthocyanidins, (OPCs/PCOs), astaxanthin, carotenoids, coenzyme Q10 (“CoQ10”), flavonoids, glutathione, Goji (Wolfberry), hesperidin, lacto-Wolfberry, lignan, lutein, lycopene, polyphenols, selenium, vitamin A, vitamin C, vitamin E, zeaxanthin, or combinations thereof.

The composition may comprise a mineral including boron, calcium, chloride, chromium, copper, iodine, iron, magnesium, manganese, molybdenum, nickel, phosphorus potassium, selenium, silicon, sodium, tin, vanadium, zinc, or combinations thereof in any suitable form, e.g., in the form of a mineral or other bioavailable complex.

The composition may comprise a phytonutrient selected from the group consisting of flavanoids, allied phenolic compounds, polyphenolic compounds, terpenoids, alkaloids, sulphur-containing compounds, or combinations thereof The phytonutrient may be selected from the group consisting of carotenoids, plant sterols, quercetin, curcumin, limonin, or combinations thereof.

The composition may comprise a herbal medicine known to have or suspected of having anti-diabetic effects. Such herbal medicine may include a medicinal plant, for example, from the Malvaceae family, such as Abelmoschus inoschatus Medik, or from the Asteraceae family, such as Achiliea santolina L., or Achyrocline satureioides (Lam.) DC, or Artemisia dracunculus L. (dragon herb) or Cichorium intybus L., or Eclipta alba (L) Hassk, or from the Lamiaceae family such as Ajuga iva L. Schreberr (Medit), or Ocimum sanctum Linn. (Tulasi), or Origanum vulgare L. or from the Annonaceae family such as Annona squamosa L., or from the Gentianeacea family such as Anthocleista djalonensis A. Chev (cabbage tree), or Anthocleista Schweinfurthii, or Anthocleista vogelii Planch, Enicostemma littorale Blume or Gentiana olivieri L., or from the Oxalidaceae family such as Averrhoa bilimbi L, or Biophytum sensitivum (L) DC, or from the Leguminosae family such as Bauhinia candicans Benth, or from the Bixaceae family such as Bixa orellana L., or from the Nyctaginaceae family such as Boerhaavia diffusa L., or from the Brassicaceae family such as Brassica nigra (L) Koch, or Eruka sativa, or Lepidium sativum L. or from the Caesalpiniaceae family such as Capparis spinosa L. or Cassia auriculata L. or Tamarindus indica L., or from the Capparidaceae family such as Capparis spinosa L., or from the Apiaceaelimily such as Carum carvi L., or from the family Rutaceae such as Clausena anisata (Wild) Benth., or from the family Palmae such as Cocos nucifera Linn. (Coconut palm), or from the Cucurbitaceae family such as Cogniauxia podoleana, or Ibervillea sonorae S., or from the family Conimelinaceae such as Commelina communis L., or from the family Zingiberaceae such as Curcuma longa L., or from the family Poaceae such as Cynodon dactylon Pers. (Bermuda grass), or from the family Ginkgoaceae such as Ginkgo biloba L., or from the family Papilionaceae such as Glycyrrhiza uralensis Fish., or from the family Asclepiadaceae, such as Gongronema lantifilium Benth., or Gymnema montanum Hook, or from the family Sterculiaceae such as Helicteres isora L., As., or from the Rubiaceae family such as Hintonia standleyana, or from the Gramineae family such as Hordeum vulgare L. (Barley), or from the Convolvulaceae family such as Ipomoea aquatic Forsk., or Ipomea batata Linn (Sweet potato), or from the Loranthaceae family such as Loranthus micranthus Linn, or from the Moraceae family such as Morus indica. L., or from the Musaceae family such as Musa sapientum Kuntz (Banana), or from the Euphorbiaceae family such as Phyllanthus amarus Schum. Thonn, or Phyllanthus niruri L., or Phyllanthus sellowianus Mull. Arg., or from the Piperacea family such as Piper longum, or from the Myrtaceae family such as Psidium guajava L., or Syzygium alternifolium (Wt) Walp, or from the Lythraceae family such as Punica granatum L. (pomegranate), or from the Papilionaceae family such as Retama raetam (RR) (Forssk) Webb., or from the Adoxaceae family such as Sambucus nigra L., or from the Apocynaceae family such as Sanguis draxonis, or from the Anacardiaceae family such as Sclerocarya birea (A. Rich), or from the Scrophariaceae family such as Scoparia dulcis L., or from the Caryophyllaceae family such as Spergularia purpurea, or from the Chenopodiaceae family such as Suaeda fruticosa (SF) Euras, or from the Combretaceae family such as Terminalia chebula Retz., or Terminalia bellirica (Gaertn), or from the Menispermaceae family such as Tinospora cordifolia Miers., or from the Urticaceae family such as Urtica pilulifera L., or from the Astereaceae family such as Vernonia amygdalina Del., or from the Solanaceae family such as Withania soimifera (L) Dunal, or from the Zygophyllaceae family such as Zygophyllum gaetulum Emb and Maire.

The composition may comprise one or more vitamins and/or amino acids. The vitamins may be selected from, but not limited to: vitamins A, B₁, B₂, B₃, B₅, B₆, B₉, B₁₂, C, D, E, K, K1 and K2 (i.e. MK-4, MK-7), folic acid or biotin. The amino acids may be selected from, but are not limited to: alanine, arginine, aspartic acid, cystine, citrulline, glycine, glutamate, glutamine, histidine, hydroxyproline, hydroxyserine, hydroxytyrosine, hydroxylysine, isoleucine, lysine, methionine, phenylalanine, proline, serine, taurine, threonine, tryptophan tyrosine, valine, or ornithine.

The composition may comprise a source of omega-3 or omega-6 fatty acids, for example, fish oil, krill, plant sources of omega-3, flaxseed, canola oil, walnut, and algae. Examples of omega-3 fatty acids include, for example, omega-linolenic acid (“ALA”), docosahexaenoic acid (“DHA”), stearidonic acid (SDA), eicosapentaenoic acid (“EPA”), or combinations thereof. Examples of omega-6 fatty acids include linoleic acid (“LA”), arachidonic acid (“ARA”). The omega-3 fatty acids may be provided in an amount of about 0.25 g to 5.0 g per day, such as from about 1.0 to 3.0 g per day.

The composition may comprise a phenolic compound, including, for example, monophenols (such as, for example, apiole, carnosol, carvacrol, dillapiole, rosemarinol); flavonoids (polyphenols) including flavonols (such as, for example, quercetin, fingerol, kaempferol, myricetin, rutin, isorhamnetin), flavanones (such as, for example, fesperidin, naringenin, silybin, eriodictyol), flavones (such as, for example, apigenin, tangeritin, luteolin), flavan-3-ols (such as, for example, catechins, (+)-catechin, (+)-gallocatechin, (−)-epicatechin, (−)-epigallocatechin, (−)epigallocatechin gallate (EGCG), (−)-epicatechin 3-gallate, theaflavin, theaflavin-3-gallate, theaflavin-3′-gallate, theaflavin-3,3′-digallate, thearubigins), anthocyanins (flavonals) and anthocyanidins (such as, for example, pelargonidin, peonidin, cyanidin, delphinidin, malvidin, petunidin), isoflavones (phytoestrogens) (such as, for example, daidzein (for mononetin), genistein (biochanin A), glycitein), dihydroflavonols, chalcones, coumestans (phytoestrogens), and Coumestrol; Phenolic acids (such as: Ellagic acid, Gallic acid, Tannic acid, Vanillin, curcumin); hydroxycinnamic acids (such as, for example, caffeic acid, chlorogenic acid, cinnamic acid, ferulic acid, coumarin); lignans (phytoestrogens), silymarin, secoisolariciresinol, pinoresinol and lariciresinol); tyrosol esters (such as, for example, tyrosol, hydroxytyrosol, oleocanthal, oleuropein); stilbenoids (such as, for example, resveratrol, pterostilbene, piceatannol) and punicalagins; terpenes (isoprenoids) including carotenoids (tetraterpenoids) including carotenes (such as, for example, beta-carotene, [3-carotene, gama-carotene, delta-carotene, lycopene, neurosporene, phytofluene, phytoene), and xanthophylls (such as, for example, canthaxanthin, cryptoxanthin, aeaxanthin, astaxanthin, lutein, rubixanthin); monoterpenes (such as, for example, limonene, perillyl alcohol); saponins; lipids including: phytosterols (such as, for example, campesterol, beta sitosterol, gamma sitosterol, stigmasterol), tocopherols (vitamin E), and omega-3, 6, and 9 fatty acids (such as, for example, gamma-linolenic acid); triterpenoid (such as, for example, oleanolic acid, ursolic acid, betulinic acid, moronic acid); betalains, including betacyanins (such as: betanin, isobetanin, probetanin, neobetanin); and betaxanthins (non glycosidic versions) (such as, for example, indicaxanthin, and vulgaxanthin); organosulfides, including, for example, dithiolthiones (isothiocyanates) (such as, for example, sulphoraphane); and thiosulphonates (allium compounds) (such as, for example, allyl methyl trisulfide, and diallyl sulfide), indoles, glucosinolates, including, for example, indole-3-carbinol; sulforaphane; 3,3′-diindolylmethane; sinigrin; allicin; alliin; allyl isothiocyanate; piperine; syn-propanethial-S-oxide; protein inhibitors, which include, for example, protease inhibitors; other organic acids including oxalic acid, phytic acid (inositol hexaphosphate); tartaric acid; and anacardic acid; or combinations thereof.

The composition may comprise a protein. Non-limiting examples of proteins include dairy based proteins, plant based proteins, animal based proteins and artificial proteins. Dairy based proteins may be selected from the group consisting of casein, micellar casein, caseinates, casein hydrolysate, whey, whey hydrolysates, whey concentrates, whey isolates, milk protein concentrate, milk protein isolate, or combinations thereof. Plant based proteins may include, for example, soy protein (e.g., all forms including concentrate and isolate), pea protein (e.g., all forms including concentrate and isolate), canola protein (e. g., all forms including concentrate and isolate), wheat and fractionated wheat proteins, corn and it fractions including zein, rice, oat, potato, peanut, and any proteins derived from beans, buckwheat, lentils, pulses, single cell proteins, or combinations thereof. Animal based proteins may be selected from the group consisting of beef, poultry, fish, lamb, seafood, or combinations thereof.

The composition may comprise a source of phytochemicals, e.g., flavonoids and allied phenolic and polyphenolic compounds, terpenoids, e.g., carotenoids and plant sterols and alkaloids and sulfur containing compounds.

The composition may comprise a source of carbohydrates. Any suitable carbohydrate may be used, including, but not limited to, sucrose, lactose, glucose, fructose, corn syrup solids, maltodextrin, modified starch, amylose starch, tapioca starch, corn starch or combinations thereof.

The composition may comprise grains. The grains may include, for example, whole grains, which may be obtained from different sources. The different sources may include semolina, cones, grits, flour and micronized grain (micronized flour), and may originate from a cereal or a pseudo-cereal. The grain may be a hydrolysed whole grain component, i.e., an enzymatically digested whole grain component or a whole grain component digested by using at least an alpha-amylase, which alpha-amylase shows no hydrolytic activity towards dietary fibers when in the active state. The hydrolsed whole grain component may be further digested by the use of a protease, which protease shows no hydrolytic activity towards dietary fibers when in the active state. The hydrolyzed whole grain component may be provided in the form of a liquid, a concentrate, a powder, a juice, a puree, or combinations thereof.

The composition may comprise a source of fat. The source of fat may include any suitable fat or fat mixture. For example, the fat source may include, but is not limited to, vegetable fat (such as olive oil, corn oil, sunflower oil, high-oleic sunflower, flax seed oil, rapeseed oil, canola oil, high oleic canola oil, hazelnut oil, soy oil, palm oil, coconut oil, blackcurrant seed oil, borage oil, lecithins, and the like), animal fats (such as milk fat), or combinations thereof. The source of fat may also be less refined versions of the fats listed above (e.g., olive oil for polyphenol content).

Where the composition is formulated as an oral supplement, it may contain Pima pinaster, Aloe vera and papain in addition to an appropriate nutritional profile that contains 10 or more grams of high quality protein, which may be provided as whey protein micelle, lipids with the EPA and DHA, and carbohydrates for energy and palatability. Vitamins such as vitamin D and minerals and ingredients such as lactowolfberry may also be included.

The composition may comprise a probiotic strain.

The composition may comprise all three branched chain amino acids, leucine, isoleucine, and valine.

### Composition Form

The composition according to the present invention may be formulated for administration in any suitable manner. For example, the composition may be formulated for oral administration, e.g., as a read-to-drink liquid mixture, solution, suspension, emulsion, syrup or in a liquid comestible such as a soft drink, juice, sports drink, milk drink, yogurt drink, soup, etc. The composition may be an aqueous composition. Alternatively, the composition may be formulated for oral administration in the form of a tablet, capsule, chewable tablet, or soft gel capsule.

In one embodiment, the composition disclosed herein is an aqueous composition comprising: 2.40 mg/mL papain; 2.60 mg/mL dry Pinus pinaster stem bark extract; 1.75 mg/mL dry Aloe vera inner leaf juice; 100.00 mg/mL honey; 2.00 mg/mL glacial acetic acid; 3.00 mg/mL sodium chloride; 400 μg/mL potassium sorbate; 400 μg/mL sodium benzoate; 10.00 mg/mL wildberry flavour UA71225; and 800 μg/mL anthocyanin extract.

In another embodiment, the composition disclosed herein is aqueous composition comprising papain, dry Pinus pinaster stem bark extract, dry Aloe vera inner leaf juice, honey, glacial acetic acid, sodium chloride, potassium sorbate, sodium benzoate, wildberry flavour UA71225 and anthocyanin extract in a ratio of papain:dry Pinus pinaster stem bark extract:dry Aloe vera inner leaf juice:honey:glacial acetic acid:sodium chloride:potassium sorbate : sodium benzoate:wildberry flavour UA71225:anthocyanin extract of 2.40:2.60:1.75:100:2.00:3.00:0.40:0.40:10.00:0.80 by weight. As such, compositions comprising the same ingredients in the same relative proportions but in different total volumes are contemplated, e.g., concentrated or dilute aqueous compositions having the same relative proportions of ingredients.

The composition disclosed herein, in particular the aqueous compositions disclosed herein, may have a pH of between about 3.2 and about 4.2, e.g., a pH of about 3.2, about 3.5, about 3.7, about 3.9, or about 4.1, or may be adjusted so as to have a pH of between about 3.2 and about 4.2, e.g., a pH of about 3.2, about 3.5, about 3.7, about 3.9, or about 4.1. In one embodiment, the composition disclosed herein, in particular the aqueous compositions disclosed herein, has a pH of about 3.7.

A further alternative formulation contemplated in the invention is provision of the composition in dry form as a powder or granules, or as a dry or liquid concentrate, which can then be diluted with water or another liquid, such as milk or fruit juice, to yield a ready-to-drink composition. The powder or granules may be provided in a sachet, which is optionally soluble. Alternatively, the composition may be consumed as a powder in the absence of a drink or additional food product. The composition may be assembled and packaged as a powder for dissolution at the time of use.

Where the composition is provided in dry form, e.g., in the form of powder or granules, the composition may comprise a therapeutically effective amount of Pinus pinaster stem bark extract corresponding to a daily adult human dosage of from about 60 mg to about 1500 mg Pinus pinaster stem bark extract; a therapeutically effective amount of papain corresponding to a daily adult human dosage of from about 30 mg to about 1200 mg papain; and a therapeutically effective amount of Aloe vera extract corresponding to a daily adult human dosage of from about 15 mg to about 600 mg Aloe vera extract. In one embodiment, a dry form of the composition disclosed herein comprises a therapeutically effective amount of Pinus pinaster stem bark extract corresponding to a daily adult human dosage of about 260 mg; a therapeutically effective amount of papain corresponding to a daily adult human dosage of about 240 mg; and a therapeutically effective amount of Aloe vera extract corresponding to a daily adult human dosage of about 175 mg.

The composition described herein may be added to a food or beverage such as a water-based beverage, a milk-based beverage, a yoghurt-based beverage, another dairy-based beverage, a milk-substitute based beverage such as soy milk or oat milk, or a juice-based beverage, water, a soft drinks, a carbonated drink, or a nutritional beverage, (including a concentrated stock solution of a beverage and a dry powder for preparation of such a beverage), or in a baked product such as a cracker, a bread, a muffin, a roll, a bagel, a biscuit, a cereal, a bar such as a muesli bar, a health food bar and the like, a dressing, a sauce, a custard, a yoghurt, a pudding, a pre-packaged frozen meal, a soup or a confectionery item.

The composition according to the present invention may be formulated for oral administration using any suitable method known in the art using suitable excipients, diluents and fillers. In general, oral compositions are prepared by uniformly and intimately bringing into association the components of the composition with a liquid carrier or finely divided solid carrier, or both and then, if necessary, shaping the product into the desired composition.

For example, when the composition is formulated as capsules, the components of the composition may be formulated with one or more pharmaceutically acceptable carriers such as starch, lactose, microcrystalline cellulose and/or silicon dioxide. Additional ingredients may include lubricants such as magnesium stearate and/or calcium stearate. The capsules may optionally be coated, for example, with a film coating or an enteric coating and/or may be formulated so as to provide slow or controlled release of the composition therein.

Tablets may be prepared by compression or moulding, optionally with one or more additional ingredients. Compressed tablets may be prepared by compressing in a suitable machine the components of the composition in a free-flowing form such as a powder or granules, optionally mixed with a binder, lubricant (for example magnesium stearate or calcium stearate), inert diluent or a surface active/dispersing agent. Moulded tablets may be made by moulding a mixture of the powdered composition moistened with an inert liquid diluent, in a suitable machine. The tablets may optionally be coated, for example, with a film coating or an enteric coating and/or may be formulated so as to provide slow or controlled release of the composition therein.

If the compositions are formulated to be administered orally, the compositions may be a liquid oral supplement. Such liquid oral supplements may comprise additional beneficial ingredients, for example, as outlined in the section herein entitled ‘Additional Components’, which may allow for more efficient use of the administered composition for preservation of regulated blood glucose levels.

### Methods of Treatment and Uses

The composition of the invention as described herein is suitable for treatment and prevention of a variety of conditions associated with or caused by elevated blood sugar. For example, the composition may be suitable for treating and/or preventing any dysfunction associated with excess blood glucose, which may include, for example, disturbances in everyday physiological functions such as increased thirst and hunger, frequent urination, fatigue/lethargy blurry vision, sleepiness, and muscle weakness. An associated dysfunction may result from, result in, be characterised by, or otherwise be associated with a high blood glucose level in a subject and may directly or indirectly result from the high blood glucose level. In some cases, the associated dysfunction may be temporally separated from when the high blood glucose level is recorded. As high blood glucose levels may lead to disturbances in overall physiological function and may induce or promote a chronic disease, reducing blood glucose levels to a normal range may be beneficial in reducing the risk of metabolic disorders, and thus treatment of high blood glucose levels may also treat chronic diseases or symptoms thereof. Accordingly, methods of treating chronic diseases associated with elevated blood glucose levels or symptoms thereof are also contemplated herein.

Accordingly, methods for stimulating regulation of high blood glucose levels in a subject in need of same are provided herein. Methods for alleviating or minimising the adverse effects of high blood glucose levels in a subject in need of same are provided. Methods for preserving a blood glucose level in the normal physiological range in a subject in need of same are provided. The methods include administering to the individual a composition as described in the section herein entitled ‘COMPOSITION’. The compositions of the present disclosure may also include other active or inactive ingredients as discussed herein.

For example, the present invention provides a method of treating or preventing elevated blood glucose, comprising administering to a subject in need thereof an effective amount of a composition as described in the section herein entitled ‘COMPOSITION’. There is also provided a method of treating or preventing pre-diabetes, comprising administering to a subject in need thereof an effective amount of a composition as described in the section herein entitled ‘COMPOSITION’.

There is further provided a method of reducing inflammation, comprising administering to a subject in need thereof an effective amount of a composition as described in the section herein entitled ‘COMPOSITION’. The inflammation may be, for example, in an athlete, and in particular, an athlete undergoing intense exercise/training or endurance exercise. There is still further provided a method of decreasing cytokine-induced inflammation, or of decreasing oxidative stress responses, comprising administering to a subject in need thereof an effective amount of a composition as described in the section herein entitled ‘COMPOSITION’. There is also provided a method of treating or preventing a disease characterised by elevated levels of cytokine-induced inflammation, or characterised by elevated levels of oxidative stress, comprising administering to a subject in need thereof an effective amount of a composition as described in the section herein entitled ‘COMPOSITION’. The disease may be atherosclerosis, liver inflammation associated with type 2 diabetes and obesity, endometriosis, or osteoarthritis.

There is also provided a method of reducing one or more side effects, in particular, adverse side effects, of a diabetes medication selected from the group consisting of metformin, sulfonylureas, meglitinides, thiazolidinediones, DPP-4 inhibitors, GLP-1 receptor agonists, SGLT2 inhibitors, and insulin therapy, comprising administering to a subject in need thereof an effective amount of a composition as described in the section herein entitled ‘COMPOSITION’. Common adverse side effects may include abdominal or stomach discomfort, decreased appetite, or diarrhoea.

There is further provided a method of treating or preventing a dysfunction associated with excess blood glucose, comprising administering to a subject in need thereof an effective amount of a composition as described in the section herein entitled ‘COMPOSITION’. The dysfunction may be increased thirst and hunger, frequent urination, fatigue/lethargy, blurry vision, sleepiness, or muscle weakness, or a combination thereof.

The composition of the present invention may be administered in accordance with the methods described herein alone, or in combination with metformin or one or more other blood glucose lowering medication(s).

Still further, there is provided a method of improving organ function comprising administering to a subject in need thereof an effective amount of a composition as described in the section herein entitled ‘COMPOSITION’. The term “improving organ function” means that a composition is administered to, or a method is used for, a subject for a period effective to improve function as determined by comparison with function in subjects not being administered the composition or using the method or determined by comparison with function in subjects prior to being administered the composition or using the method. Any suitable method of assessing organ function can be used to determine whether an improvement occurs. In one embodiment, organ function is assessed by the level(s) of one or more biomarkers in a tissue or fluid sample, for example systemic inflammation content of blood. Elevated levels in blood of human C-Reactive Protein (CRP), in particular the slightly to moderately elevated levels are an indicator of when there is inflammation in the body.

The composition of the invention as described herein is suitable for treating and regulating high cholesterol levels. In particular, the composition of the invention may be suitable for lowering blood cholesterol in subjects in need thereof. Accordingly, there is provided a method of lowering blood cholesterol comprising administering to a subject in need thereof an effective amount of a composition as described in the section herein entitled ‘COMPOSITION’.

The blood cholesterol may be selected from the group consisting of: low density lipoproteins (LDL), high density lipoproteins (HDL), trigylcerides and total cholesterol, or a combination thereof. For example, the composition may be used to lower low density lipoprotein (LDL) or ‘bad’ blood cholesterol levels in a subject, may additionally or alternatively be used to increase high density lipoprotein (HDL) or ‘good’ blood cholesterol levels, or may be used to decrease blood triglyceride levels. The composition may be used to decrease the ratio of Total Cholesterol to HDL in the blood of a subject.

The term ‘increasing’ and related terms ‘increase’, ‘increased’ and the term ‘decreasing’ and related terms ‘decrease’, ‘decreased’ mean that a composition is administered to, or a method is used for, a subject for a period effective to change the level of the recited marker, e.g., HDL or LDL cholesterol, as determined by comparison with the levels of the recited marker in subjects not being administered the composition or in subjects not using the method, or determined by comparison with a level of the marker in a subject prior to the subject being administered the composition or prior to using the method. Any suitable method of assessing levels of the recited marker, e.g., cholesterol, may be used to determine whether an increase or decrease occurs, and methods for measuring blood cholesterol are known in the art.

The composition of the invention as described herein is also suitable for improving immune function, and accordingly, a method of improving immune function in a subject in need thereof by administering an effective amount of a composition as described in the section herein entitled ‘COMPOSITION’ is also provided herein. Improved immune function may be measured using any suitable immune function indicator, for example, as an increased white blood cell count, where the white blood cells are, e.g., neutrophils, using methods known in the art.

The composition of the invention as described herein is further suitable for treating an autoimmune disease, and accordingly, methods of treating an autoimmune disease comprising administering to a subject in need thereof an effective amount of a composition as described in the section herein entitled ‘COMPOSITION’ are also provided herein. The autoimmune disease may be any autoimmune disease, for example, it may be selected from the group consisting of: lupus, arthritis, psoriasis, and type I diabetes. Where the autoimmune disease is lupus, the treating may include decreasing the number and/or severity of lesions, e.g., sub cutaneous lesions, in or on the subject. Where the autoimmune disease is arthritis, in particular rheumatoid arthritis, the treating may include decreasing swelling and pain in the subject. Where the autoimmune disease is psoriasis, the treating may include reducing the number and/or severity of skin plaques, decreasing swelling or inflammation, and/or decreasing pain. Where the autoimmune disease is type I diabetes, the treating may enable a reduction in the amount of insulin otherwise required for effective management of blood sugar levels.

The composition of the invention as described herein may also be used to treat emphysema, and accordingly, there is provided a method of treating emphysema comprising administering to a subject in need thereof an effective amount of a composition as described in the section herein entitled ‘COMPOSITION’.

The composition of the invention as described herein may be used to increase general wellbeing in a subject, and accordingly, there is provided a method of increasing general wellbeing comprising administering to a subject in need thereof an effective amount of a composition as described in the section herein entitled ‘COMPOSITION’. A measure of increased general wellbeing may include one or more of: increased energy levels, better sleep, improved breathing ability, improved levels of physical activity, and/or improved physical functioning.

The composition as described herein may have potent antioxidant activity and anti-inflammatory actions and support a healthy immune system. For example, it may have excellent radical scavenging properties and may support the health and function of the immune system. These properties may cause or assist the observed therapeutic effects of the compositions described herein for a range of diseases and conditions.

Also contemplated herein are uses of a composition as described in the section herein entitled ‘COMPOSITION’ for the manufacture of a medicament for: treating or preventing elevated blood glucose; or preventing or delaying the onset of type 2 diabetes mellitus (T2DM); or treating or preventing pre-diabetes; or reducing inflammation; or decreasing cytokine-induced inflammation; or decreasing an oxidative stress response; or treating or preventing a disease characterised by elevated levels of cytokine-induced inflammation; or treating or preventing a disease characterised by elevated levels of oxidative stress; or reducing one or more side effects of a diabetes medication selected from the group consisting of metformin, sulfonylureas, meglitinides, thiazolidinediones, DPP-4 inhibitors, GLP-1 receptor agonists, SGLT2 inhibitors, and insulin therapy; or treating or preventing a dysfunction associated with excess blood glucose; or improving organ function; or lowering blood cholesterol; or improving immune function; or treating an autoimmune disease, e.g., lupus, arthritis, psoriasis, or type I diabetes; or treating emphysema; or increasing general wellbeing, e.g., increasing energy levels, improving vitality, improving sleep, improving breathing ability, improving levels of physical activity, improving mental functioning and/or improving physical functioning in a subject.

In the methods and uses described herein, the subject may be elderly, have a medically diagnosed or undiagnosed condition of pre-diabetes, or be at risk of developing pre-diabetes. The subject may be taking metformin or other blood glucose lowering medications at the time of taking the composition described herein, or may have been treated with metformin or other blood glucose lowering medications prior to taking the composition.

In the methods and uses described herein, the terms “treating”, “treatment” and the like refer to any and all applications which remedy, or otherwise hinder, retard, or reverse the progression of, a disease or at least one symptom of a disease, including reducing the severity of a disease. Thus, treatment does not necessarily imply that a subject is treated until complete recovery from a disease. Similarly, the terms “preventing”, “prevention” and the like refer to any and all applications that prevent the establishment of a disease or otherwise delay the onset of a disease.

The composition described herein may further comprise one or more additives or additional components as described in the sections herein entitled ‘Additives’ and ‘Additional Components’ respectively such that the composition may provide other health benefits, for example, assisting with muscle protein synthesis.

### Dosages

In general, the composition described herein may be administered in any suitable dose amount that is effective as a health supplement and/or as therapeutic agent to achieve the desired health outcome. Those skilled in the art will appreciate that single or multiple administrations of the composition disclosed herein can be carried out with dose levels and dosing regimens being determined as required depending on the circumstances and on the condition of the subject to be treated. The skilled addressee can readily determine suitable dosage regimes. A broad range of doses may be applicable. Dosage regimens may be adjusted to provide the optimum therapeutic response. Those skilled in the art will appreciate that the exact amounts and rates of administration of the composition disclosed herein will depend on a number of factors such as the particular composition being administered, the age, body weight, general health, sex and dietary requirements of the subject, as well as any drugs or agents used in combination or coincidental with the composition. For example, several divided doses may be administered hourly, daily, weekly, monthly or at other suitable time intervals or the dose may be proportionally reduced as indicated by the exigencies of the situation. Based on the teaching herein, those skilled in the art will, by routine trial and experimentation, be capable of determining suitable dosage regimes on a case-by-case basis.

Compositions and methods of the present disclosure may be employed as an adjunct to other therapies or treatments for chronic diseases such as pre-diabetes, diabetes and conditions associated therewith. Accordingly, compositions and methods disclosed herein may be co-administered with other agents that may facilitate a desired therapeutic outcome, for example, one more renal drugs, or may be sequentially administered with other agents, for example, other blood-sugar regulating or diabetes medications, such as insulin. By “co-administered” is meant simultaneous administration in the same formulation or in two different formulations via the same or different routes or sequential administration by the same or different routes. By “sequential” administration is meant a time difference of from seconds, minutes, hours or days between the administration of the agents, compositions or treatments. Sequential administration may be in any order.

In one embodiment, the composition of the present disclosure may be provided to an adult human subject so as to deliver a total amount of Pinus pinaster stem bark extract (equivalent to dry Pinus pinaster stem bark extract) of from about 60 mg to about 1500 mg per subject per day, e.g., from about 60 mg/day to about 100 mg/day, from about 75 mg/day to about 125 mg/day, from about 100 mg/day to about 200 mg/day, from about 150 mg/day to about 250 mg/day, from about 200 mg/day to about 300 mg/day, or from about 250 mg/day to about 350 mg/day, or from about 300 mg/day to about 400 mg/day, or from about 400 mg/day to about 600 mg/day, or from about 500 mg/day to about 700 mg/day, or from about 600 mg/day to about 800 mg/day, or from about 700 mg/day to about 900 mg/day, or from about 800 mg/day to about 1000 mg/day, or from about 900 mg/day to about 1100 mg/day, or from about 1000 mg/day to about 1200 mg/day, or from about 1000 mg/day to about 1500 mg/day, such as about 60 mg/day, about 100 mg/day, about 150 mg/day, about 200 mg/day, about 220 mg/day, about 240 mg/day, about 260 mg/day, about 280 mg/day, about 300 mg/day, about 320 mg/day, about 340 mg/day, about 360 mg/day, about 380 mg/day, about 400 mg/day, about 500 mg/day, or about 600 mg/day, or about 800 mg/day, or about 1000 mg/day, or about 1200 mg/day or about 1500 mg/day. These total daily dosages may be provided in any suitable number of doses, e.g., in a single dose, or in multiple (i.e., two, three, four or more doses). The doses may comprise an equal amount of Pinus pinaster stem bark extract. For example, in one dosage regime, a subject may be administered two doses of a composition according to the invention per day, wherein each composition comprises from about 100 mg to about 200 mg of Pinus pinaster stem bark extract, e.g., from about 100 mg to about 150 mg, or from about 120 mg to about 175 mg, or from about 150 mg to about 200 mg of Pinus pinaster stem bark extract, for example, about 100 mg, 110 mg, 120 mg, 130 mg, 140 mg, 150 mg, 160 mg, 170 mg, 180 mg, 190 mg or 200 mg Pinus pinaster stem bark extract.

The composition of the present disclosure may be provided to an adult human subject so as to deliver a total amount of Pinus pinaster stem bark extract (equivalent to dry Pinus pinaster stem bark extract) of about 260 mg per subject per day, said amount being effective for treating or preventing elevated blood glucose, or preventing or delaying the onset of type 2 diabetes mellitus (T2DM), or treating or preventing pre-diabetes; or reducing inflammation, or decreasing cytokine-induced inflammation; or decreasing an oxidative stress response, or treating or preventing a disease characterised by elevated levels of cytokine-induced inflammation, or treating or preventing a disease characterised by elevated levels of oxidative stress, or reducing one or more side effects of a diabetes medication selected from the group consisting of metformin, sulfonylureas, meglitinides, thiazolidinediones, DPP-4 inhibitors, GLP-1 receptor agonists, SGLT2 inhibitors, and insulin therapy, or treating or preventing a dysfunction associated with excess blood glucose, or improving organ function, or improving immune function, or treating an autoimmune disease, e.g., lupus, arthritis, psoriasis, or type I diabetes, or treating emphysema, or increasing general wellbeing, e.g., increasing energy levels, improving vitality, improving sleep, improving breathing ability, improving levels of physical activity, improving mental functioning, and/or improving physical functioning.

The amount of Pinus pinaster stem bark extract may be increased to more than 260 mg per day depending on the severity of the disease or condition to be treated. Once the disease or condition has been effectively ameliorated, the adult subject may decrease the dosage to about 100 mg per day for maintenance purposes.

In another embodiment, the composition of the present disclosure may be provided to an adult human subject so as to deliver a total amount of papain (equivalent to dry papain) of from about 30 mg to about 1200 mg per subject per day, e.g., from about 30 mg/day to about 80 mg/day, from about 70 mg/day to about 120 mg/day, from about 100 mg/day to about 200 mg/day, from about 150 mg/day to about 250 mg/day, from about 200 mg/day to about 300 mg/day, or from about 250 mg/day to about 350 mg/day, or from about 300 mg/day to about 400 mg/day, or from about 400 mg/day to about 600 mg/day, or from about 600 mg/day to about 800 mg/day, or from about 800 mg/day to about 1000 mg/day, or from about 900 mg/day to about 1200 mg/day, such as about 60 mg/day, about 100 mg/day, about 150 mg/day, about 200 mg/day, about 220 mg/day, about 240 mg/day, about 260 mg/day, about 280 mg/day, about 300 mg/day, about 320 mg/day, about 340 mg/day, about 360 mg/day, about 380 mg/day, about 400 mg/day, about 500 mg/day, about 600 mg/day, or about 800 mg/day, or about 1000 mg/day or about 1200 mg/day. These total daily dosages may be provided in any suitable number of doses, e.g., in a single dose, or in multiple (i.e., two, three, four or more doses). The doses may comprise an equal amount of papain.

In another embodiment, the composition of the present disclosure may be provided to an adult human subject so as to deliver a total amount of Aloe vera extract (equivalent to dry Aloe vera extract, particularly equivalent to spray dried Aloe vera inner leaf juice extract) of from about 15 mg to about 600 mg per subject per day, e.g., from about 15 mg/day to about 80 mg/day, from about 50 mg/day to about 100 mg/day, from about 75 mg/day to about 150 mg/day, from about 100 mg/day to about 200 mg/day, from about 150 mg/day to about 300 mg/day, or from about 250 mg/day to about 350 mg/day, or from about 300 mg/day to about 400 mg/day, or from about 400 mg/day to about 600 mg/day, such as about 15 mg/day, about 30 mg/day, about 60 mg/day, about 90 mg/day, about 120 mg/day, about 175 mg/day, about 200 mg/day, about 250 mg/day, about 300 mg/day, about 340 mg/day, about 380 mg/day, about 400 mg/day, about 500 mg/day, or about 600 mg/day. These total daily dosages may be provided in any suitable number of doses, e.g., in a single dose, or in multiple (i.e., two, three, four or more doses). The doses may comprise an equal amount of Aloe vera extract.

When the composition according to the present invention is formulated as an aqueous composition having from about 2 mg/mL to about 10 mg/mL Pinus pinaster stern bark extract, from about 0.5 mg/mL to about 4 mg/mL Aloe vera extract, and about 1 mg/mL to about 8 mg/mL papain, a suitable total dosage of the aqueous composition may be from about 30 mL to about 150 mL per adult human subject per day, e.g., from about 30 mL to about 50 mL, or from about 50 mL to about 100 mL, or from about 100 mL to about 150 mL per subject per day, e.g., about 30 mL, 40 mL, 50 mL, 60 mL, 70 mL, 80 mL, 90 mL, 100 mL, 110 mL, 120 mL, 130 mL, 140 mL, or 150 mL. In an embodiment, from about 30 to about 100 mL or from about 30 mL to about 50 mL of a composition according to the invention is given to an adult human subject per day to maintain general health and wellbeing. In an embodiment, from about 50 mL to about 150 mL of a composition according to the invention is given to an adult human subject per day to treat a condition described herein, for example, to treat elevated blood sugar, pre-diabetes and/or their associated conditions. In an embodiment, from 50 to 100 mL of a composition according to the invention is given to an adult human subject per day to treat a condition described herein, for example, to treat elevated blood sugar, pre-diabetes and/or their associated conditions. As noted above, the total dosage may be administered to an adult human subject in one dose, or in multiple (i.e., two, three, four or more) smaller doses. In an embodiment, where a daily amount is administered as two doses, the doses may be taken one after a morning meal and one after an evening meal. In an embodiment, the doses may be equal doses, e.g., two equal doses of about 50 mL.

Accordingly, when the composition according to the present invention is formulated as an aqueous composition having from about 2 mg/mL to about 10 mg/mL Pinus pinaster stem bark extract, from about 0.5 mg/mL to about 4 mg/mL Aloe vera extract, and about 1 mg/mL to about 8 mg/mL papain, a suitable dosage may be from about 1 mL/kg/day to about 15 mL/kg/day, or from about 1 mL/kg/day to about 5 mL/kg/day, from about 3 mL/kg/day to about 7 mL/kg/day, or from about 10 mL/kg/day to about 15 mL/kg/day, e.g., about 1 mL/kg/day, about 3 mL/kg/day, about 5 mL/kg/day, about 7 mL/kg/day, about 9 mL/kg/day, about 11 mL/kg/day, about 13 mL/kg/day, or about 15 mL/kg/day.

Paediatric dosages of a composition comprising Pinus pinaster stem bark extract, Aloe vera extract and papain may be in the range of about 15% to about 90% of the daily adult human dosages listed herein.

### Manufacture

The disclosure herein further provides a method of manufacturing an aqueous composition, said method comprising combining Pinus pinaster stem bark extract, papain, and Aloe vera extract in water. As outlined in the section herein entitled Pinus pinaster stem bark extract', the Pinus pinaster stem bark extract may be added to the water as a solid or dry powder. As outlined in the section herein entitled ‘Papain’, the papain may be added to the water as a solid or dry powder. As outlined in the section herein entitled ‘Aloe vera’, the Aloe vera extract may be added to the water as a solid or dry powder. In one embodiment, the Aloe vera extract is Aloe vera leaf extract, in particular, dry inner leaf juice. The Pinus pinaster stem bark extract, papain, and Aloe vera extract may be combined in water in a single step, and dissolved or suspended in the water by mixing. Any suitable amount of water may be used.

The method may further comprise adding an acid. The acid may be an acid as described in the above section entitled ‘Acid’. In one embodiment, the acid is glacial acetic acid, and is partially diluted in water prior to adding to the Pinus pinaster stem bark extract, papain, and Aloe vera extract in water. The acid is preferably added to the composition after the Pinus pinaster stem bark extract, papain, and Aloe vera extract have been added.

The method may still further comprise adding honey. The honey may be as described in the section herein entitled ‘Honey’. In one embodiment, the honey is pre-heated prior to adding it to the water and Pinus pinaster stem bark extract, papain, and Aloe vera. In embodiments where acid is added to the composition, the honey is preferably added to the composition after the acid has been added and mixed.

In one embodiment, the method of manufacturing an aqueous composition of the invention comprises:
(a) dissolving sodium chloride and preservatives (potassium sorbate and sodium benzoate) in water to form a solution;
(b) adding papain, Pinus pinaster stern bark extract, dry Aloe vera juice and a pigment (anthocyanin extract) to the solution formed in step (a) with stirring to form a mixture;
(c) adding pre-diluted glacial acetic acid to the mixture formed in step (b); and
(d) adding a flavouring agent and pre-heated honey to the mixture formed in step (c).

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### EXAMPLES

The present invention will now be described with reference to specific examples, which should not be construed as in any way limiting

### Example 1

### Composition

A composition according to an embodiment of the present invention is given in Table 1.

TABLE 1

| | Content | | Content | |
|---|---|---|---|---|
| Component | (/mL) | | (/100 mL) | |
| Sodium chloride | 3.00 | mg | 0.3 | g |
| Papain | 2.40 | mg | 0.24 | g |
| Pinus pinaster stem bark extract, dry (Equiv. | 2.60 | mg | 0.26 | g |
| Pinus pinaster stem bark dry - 1.56 g) | | | | |
| Aloe vera inner leaf juice dry (Equiv. aloe | 1.75 | mg | 0.175 | g |
| vera leaf inner fresh - 350 mg) (Equiv. | | | | |
| hydroxyanthacene derivatives calculated as | | | | |
| barbaloin - 1.75 ng) (Equiv. maltodextrin - | | | | |
| 875 μg) | | | | |
| Potassium sorbate (Equiv. potassium - | 400 | μg | 40 | mg |
| 0.104 mg) | | | | |
| Sodium benzoate (Equiv. sodium - 0.064 mg) | 400 | μg | 40 | mg |
| Honey | 100.00 | mg | 10 | g |
| Acetic acid - glacial | 2.00 | mg | 0.2 | g |
| Wildberry flavour UA71225 | 10.00 | mg | 1 | g |
| Anthocyanin extract (Equiv. maltodextrin - | 800 | μg | 80 | mg |
| 280 μg) | | | | |
| Water - purified | 912.00 | mg | 91.2 | g |

### Example 2

### Sample Synthesis

An example synthesis of 100 mL of the composition given in Example 1 is as follows: Sodium chloride (0.3 g), potassium sorbate (40 mg) and sodium benzoate (40 mg) were dissolved in water (11.90 g). To the resultant solution, papain (0.24 g; Papaina (USP), Biocon Limited, Bangalore, India), Pinus pinaster stem bark extract (0.26 g; Pycnogenol®, Horphag Research USA Inc. (procyanidins 65-75%)), Aloe vera juice dry (0.175 g; Aloe barbadensis Miller, spray dried supplied by Capitol Ingredients (fresh plant:product 180-210:1; 40-50% maltodextrin)) and anthocyanin extract (80 mg; Anthocyanin extract powder, Naturex (25-35% maltodextrin, 65-75% anthocyanin extract (E163))) were added slowly, with stiffing. To this mixture, a pre-diluted solution of glacial acetic acid (0.2 g; Bronson & Jacobs Pty Limited, Sydney, Australia) in water (4.76 g) was added. To the resultant mixture, water (69.05 g), wildberry flavour (1 g; wildberry flavour UA71225, Ungerer Australia Pty Limited) and pre-heated honey (10 g; British Pharmacopoeia (BP) grade honey, Capilano Honey Limited, Australia) were added and the resultant mixture stirred until homogeneous. Further water (5.49 g) was then added and gentle stirring continued to give a brownish maroon suspension.

### Example 3

### Laboratory Studies

The results of laboratory studies exploring the effect of a composition according to the Example 1, denoted ‘Example 1’, on supressing the inflammatory and oxidative responses in cytokine-activated human coronary artery endothelial cells.

### Introduction

Chronic inflammation and oxidative stress underlies the pathogenesis of a number of lifestyle diseases including the major heart disease, atherosclerosis, and type 2 diabetes. Atherosclerosis is the major cause of mortality and morbidity in Western societies, with death rates exceeding those caused from any cancer. Type 2 diabetes is in epidemic proportions because of lifestyle imbalance, and importantly to our medical care system, type 2 diabetes increases the risk of atherosclerosis 2-4 fold, and is the leading reason for kidney failure and subsequent dependency on dialysis. Atherosclerosis is the development of lipid-filled lesions in arterial walls. The lesions develop because LDL particles accumulate in the vessel wall. In the vessel wall, the LDL particles become oxidised and in their oxidised state, the LDL particles initiate an inflammatory reaction in the cells that comprised the vessel wall. The endothelial cells that line the lumen of the artery are especially activated and in their activated state, the cells express cell adhesion molecules. These adhesion molecules bind leukocytes, including monocytes, and in the bound state, the monocytes are induced to migrate into the vessel wall. Once in the vessel wall, the monocytes differentiate into macrophages and the macrophages engulf the oxidised LDL particles. These oxidised LDL particles cause macrophage apoptosis and necrosis. The death of the macrophage causes the engulfed lipid to spill out of the cell and this coalesces with other destroyed macrophages to form the early fatty streak characteristic of an atherosclerotic lesion.

The important cell adhesion molecules that are expressed by dysfunctional endothelial cells are VCAM-1, ICAM-1 and ELAM. These are recognised markers of an inflammatory response in all cell types, but are especially important in the pathogenesis of any vascular disease. VCAM-1 and ICAM-1 are regulated by a key inflammatory mediator, nuclear factor kappa B (NFkB). If NFkB is activated then cells will increase their expression of VCAM-1 and ICAM-1.

NFkB becomes activated when cells are exposed to inflammatory cytokines and reactive oxygen species (or free radicals, ROS). Increased ROS accumulate in tissues and cells when the enzymes that produce ROS are expressed in higher levels, and are more active, than the enzymes and proteins that scavenge or metabolise the ROS. This imbalance leads to a state of cellular oxidative stress, which activates regulators such as NFkB and initiates an inflammatory response. If these responses continue unchecked the cell will enter apoptosis. A major enzyme involved in ROS production (superoxide) in endothelial cells is NADPH oxidase 4 (NOX4) while a major enzyme with antioxidant function is superoxide dismutase-1 (SOD-1).

In this study, it is reported that Example 1 was able to decrease the expression of cytokine-activated VCAM-1, ICAM-1 and ELAM in human coronary artery endothelial cells (HCAECs) via a mechanism that is associated with the decreased activation of NFkB. In keeping with the suppression of the inflammatory markers, it is reported that Example 1 decreased ROS levels in cells, with an accompanying decrease in NOX4 but increase in SOD-1, expression. The decrease in inflammatory and oxidative responses culminated in a decrease in monocyte adhesion to the endothelial cells.

### Methods

Human coronary artery endothelial cells were cultured in standard laboratory conditions. They were pre-incubated with Example 1 (50, 25, 12.5, 6.25 μl) for 3 hours before they were stimulated with the inflammatory cytokine, TNFalpha, for 1 (mRNA) or 3 (protein) hours. Cells were then assayed for cell adhesion molecule, NOX-4 and SOD-1 expression, NFkB activation, superoxide levels, and monocyte adhesion.

### Results

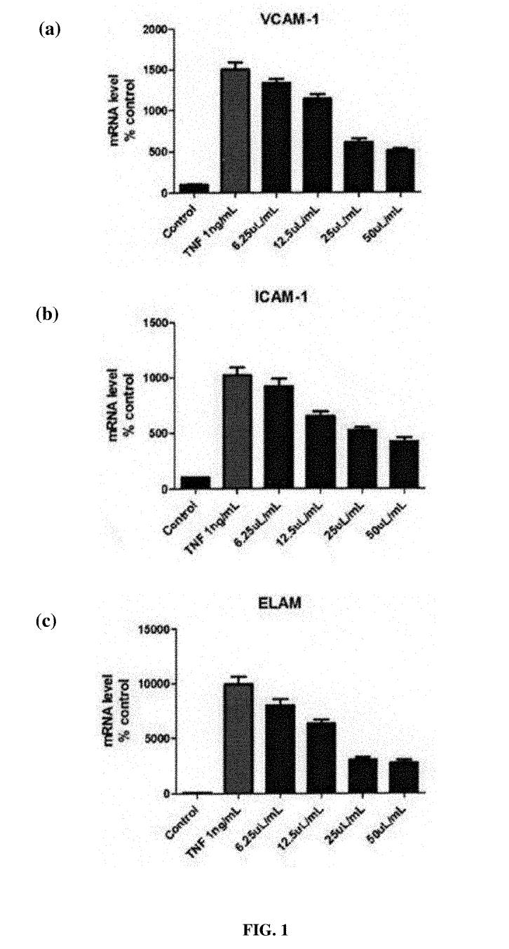

Example 1 decreases TNFalpha-induced VCAM-1, ICAM-1 and ELAM expression (see FIG. 1). HCAECs were exposed to increasing volumes of Example 1 for 3 hours. After 3 hours, HCAECs were activated with an inflammatory insult, TNFalpha for 1 hour. In HCAEC that were not exposed to Example 1, TNFalpha increased VCAM-1 expression by 15-fold, ICAM-1 by 10-fold, and ELAM by 100-fold. Example 1 supplementation was able to suppress TNFalpha induced mRNA levels for all the CAMs. The Example 1 dose of 25 uL/mL is equivalent of consuming 100 mL of Example 1. The results suggest that Example 1 is having an anti-inflammatory effect in HCAECs.

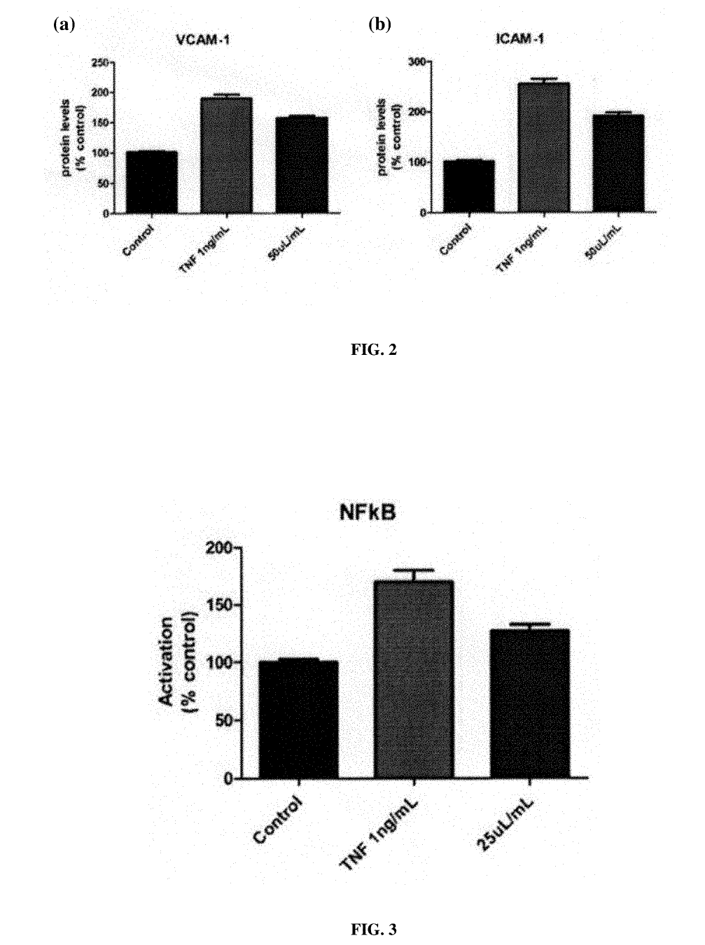

The next experiment examined if CAM protein levels were also decreased by Example 1 in the presence of inflammatory stimuli. As above, HCAECs were exposed to Example 1 for 3 hours and then activated with TNFalpha for 3 hours. Example 1 was found to significantly decrease protein levels of both VCAM-1 and ICAM-1 (FIG. 2, ELAM was not measured). Due to the half-life of membrane proteins, the effective suppression of Example 1 was not as great as for the mRNA, which has a much quicker turnover.

The expression of VCAM-1, ICAM-1 and HAM are regulated by NFkB. Therefore, it was next determined if Example 1 decreased the activation of NFkB. As shown in FIG. 3, Example 1 decreases activation of NFkB.

NFkB is a redox sensitive protein. Increased ROS levels will therefore activate this regulatory factor. The next experiment explored whether Example 1 was able to decrease ROS accumulation in human coronary artery endothelial cells. To this end, HCAECs were exposed to Example 1 for 3 hours before being loaded with the oxidant-sensitive fluorescent probe 2′7′-dichlorofluorescin (DCFH). The cells were then activated with TNFalpha for 15 rains. DCFH can be rapidly oxidised to a highly fluorescent compound called dehydrodichlorofluorescin by various ROS and peroxynitrite and it was determined that after TNFalpha activation, fluorescence intensity increased dramatically. This effect was significantly abrogated in HCAECs that were pre-exposed to Example 1. FIG. 4 shows that Example 1 decreases cytokine-induced ROS levels in HCAECs.

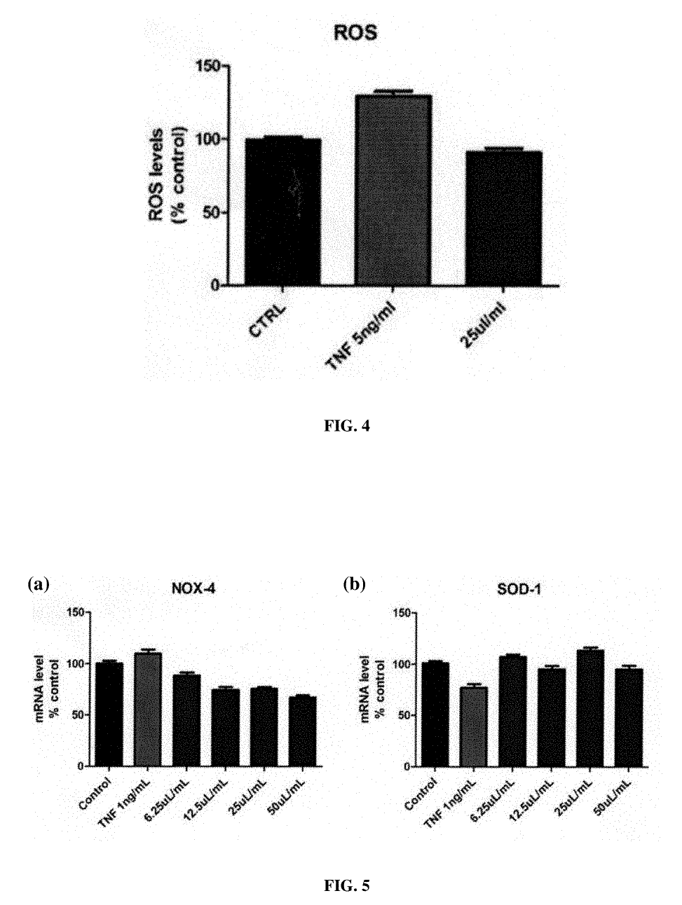

Given the finding that Example 1 decreased ROS levels within TNFalpha-activated HCAECs, it was next investigated if Example 1 modulated the expression of genes encoding for the ROS generating enzyme, NOX4 and the ROS metabolising enzyme, SOD-1. Results show that Example 1 decreased NOX4 mRNA levels by a small but significant level. Example 1 was shown to restore SOD-1 mRNA levels back to baseline. In FIG. 5, Example 1 is shown to decrease NOX-4 and increase SOD-1 levels in HCAECs.

### Conclusions

Example 1 decreases cytokine-induced inflammatory and oxidative stress responses in human coronary artery endothelial cells. The mechanism involves suppressing the activation of the key mediator of inflammation, NFkB, by decreasing the generation of ROS and thereby lowering the level of oxidative stress in HCAECs in response to inflammatory stimuli. As the onset of type 2 diabetes is driven by inflammation and oxidative stress, increases in blood levels of glucose, triglycerides, cholesterol and other toxic substances associates with diabetes leads to the deposit of triglycerides/cholesterol in the liver, and fat tissue deposition leads to inflammation of the liver, inflammation may drive the onset of insulin resistance, and if left unchecked, type 2 diabetes. More generally, inflammation and oxidative stress underlies a number of chronic diseases including atherosclerosis, liver inflammation associated with type 2 diabetes and obesity, endometriosis, and osteoarthritis. The laboratory findings for Example 1 suggest that this supplement may decrease the symptoms and causes associated with these chronic diseases.

### Example 4

### Case Study 1

Two overweight middle-aged subjects in their mid-to-late 50's diagnosed with elevated blood glucose levels were administered a liquid preparation of the composition according to Example 1. This first person, 58 years old, was overweight and diagnosed as type 2 diabetic with high blood glucose levels. After one month at a dose of 100 mL/day for 6 months, this subject presented with improved (lowered) blood glucose levels. The second person, in their mid 50's and also overweight with high blood glucose saw, after one month at a dose of 100 mL/day, a reduction in blood sugar to the normal range and improved mobility/physical functioning.

### Example 5

### Case Study 2: Patient ‘AF’

A pre-diabetic/borderline female diabetic was given a daily single dose (50 mL) of the Example 1 composition over the course of two years starting from February 2012 whilst maintaining a consistent diet and exercise regime. The blood sugar results obtained (measured as fasting plasma glucose levels) are given in Table 2.

TABLE 2

| | Test result |
|---|---|
| Date | (mmol/L) |
| 12 Sep. 2011 | 5.8 |
| (before administration of composition) | |
| 21 Apr. 2012 | 5.5 |
| 26 Oct. 2013 | 5.2 |

The patient took the single dose above each day between February 2012 and August 2014, but from August 2014 to November 2014, did not take any of the composition. The fasting plasma glucose level result after this time is shown in Table 3.

TABLE 3

| | Test result |
|---|---|
| Date | (mmol/L) |
| 17 Nov. 2014 | 6.2 |

These results demonstrate a general decline in blood glucose levels when using the composition in Example 1, and an increase in blood glucose levels when not using it.

Patient AF also reported being overweight, suffering from severe arthritis in her legs, knees, wrists and thumbs, and suffering from inflammation, as well as having severe pain and swelling in her legs due to injuries sustained in a motorbike accident. Patient AF also reported recurring cellulitis in her left leg.

After taking about 50-80 mL per day of the composition of Example 1 in the morning for a period of approximately three years, Patient AF is reportedly able to walk, work full time and lead a relatively normal lifestyle. Patient AF has also reported that the cellulitis, pain and swelling are kept at bay by the composition of Example 1, with no cellulitis recurrence since taking this composition. Patient AF also reported that her blood pressure has been kept within a normal range as a result of taking the composition of Example 1.

### Example 6

### Case Study 3: Patient ‘FC’

A 34 year old overweight man diagnosed with stomach cancer was given a daily dose of 100 mL of the composition of Example 1 for a period of 2 months. Blood samples were collected approximately 4 months prior to the first dose of the composition and shortly after the last dose at 2 months. Fasting blood cholesterol results obtained from testing these two samples are given in Table 4.

TABLE 4

| | Concentration in | Concentration in |
|---|---|---|
| | sample (mg/dl) | sample (mg/dl) |
| | 4 months prior | after 2 months |
| | to taking Example | of taking Example |
| Blood component | 1 composition | 1 composition |
| Cholesterol | 275 | 173 |
| Triglycerides | 375 | 253 |
| HDL | 55 | 45 |
| LDL | 145 | 77 |

As shown in Table 4, cholesterol, triglycerides, HDL and LDL reading have all reduced significantly after taking the composition of Example 1. The patient also reported a reduction in the swelling in his ankles after taking the composition of Example 1.

Patient FC also reported generally feeling unwell, having gout attacks, high liver enzymes and low energy levels. After 3 months of taking the composition of Example 1, along with diet changes, Patient FC reported a reduction in his weight of about 3 kg in two months. Previous weight loss from diet changes alone only resulted in a loss of 4 kg over a period of approximately four months.

Patient FC also reported that dry redness in his eyes late at night was reduced after taking the composition of Example 1, along with tinges of blood in his nose disappearing and thick build-up of greenish mucus disappearing indicative of less dryness in the area.

Patient FC further reported that numbness in his arm has gone, which discussion with a doctor indicates could be a result of the reduction in his blood cholesterol. Uncontrollable dandruff and inflamed scalp conditions have also improved significantly since taking the composition of Example 1. Shedding and inflammation of skin around and inside of his ear lobe has also reportedly gone.

With small diet changes and after taking the composition of Example 1 for 3 months, Patient FC reported that his blood work shows balancing of lipids in the blood, substantially reduced liver enzymes, and reduction in blood sugar to normal levels. He also reported substantially improved general energy and focus/concentration, especially when pain in his joints goes away.

### Example 7

### Athlete Study

### Introduction

Nitric oxide is a powerful vasodilator (dilates the blood vessels in the body) but it also has a number of other very beneficial roles on skeletal muscles. Nitric oxide is produced from arginine, an amino acid obtained from the diet from dairy products, beef, pork, poultry, gelatin, seafood, wheat germ, oatmeal, granola, peanuts and other nuts, seeds, chick peas and soybeans. Arginine is converted to nitric oxide by an enzyme called nitric oxide synthase. In blood vessels, the enzyme is called endothelial nitric oxide synthase, abbreviated to eNOS.

If a healthy diet is consumed, there will be ample arginine in the diet. What is in limited amounts in the body is the enzyme, eNOS. Therefore, only so much nitric oxide can be generated at any one time because the enzyme is in rate limiting amounts.

When the composition of Example 1 is consumed, it acts on the endothelial cells in the blood vessels to switch on the synthesis of eNOS. As there is more eNOS then it is possible to produce more nitric oxide. Moreover, the composition activates eNOS—it makes the enzyme work faster so nitric oxide is generated more rapidly. This is very important for the active athlete.

Nitric oxide has the ability to dilate blood vessels. Dilated blood vessels allow more efficient nutrient delivery to active muscles and other tissues. More efficient blood supply will also allow higher levels of oxygen to be transported at a quicker rate to tissues, including our active muscles. Dilated blood vessels also include the venous system which allows faster elimination of toxic waste products from the body.

Nitric oxide is important during exercise because it is able to boost the function of mitochondria in cells, including those in skeletal muscle cells. Mitochondria are the energy generating units in cells. The substrate that goes into mitochondria are simple sugars such as glucose and these are converted into the energy unit, ATP. Nitric oxide boosts the speed at which glucose is converted to ATP and it facilitates the entry of glucose into the cell. The faster glucose can be taken up by cells, the faster it can be converted into ATP. NO is a natural stimulant of muscle growth and repair. This will benefit the active athlete increasing strength and endurance.

Inflammation is a natural consequence of endurance exercise. The production of pro-inflammatory cytokines such as IL-1β, TNF-α, IFN-γ, is increased during intense and prolonged exercise (MacKinnon, 1999). The inflammatory cytokines will cause damage within skeletal muscle (Malm, 2001). It has been demonstrated that if blood is sampled 30 min after an Ironman event, TNF-α levels rise 3.5-fold, IFN-γ levels rise by 16.5-fold and IL-1β levels increase by 7-fold and all of these cytokines stay high for the next 48 hours, damaging muscle fibres.

Pro-inflammatory cytokines increase the stress hormones, catecholamines and corticosteroids. This is best demonstrated by the dramatic rise in cortisol levels by endurance exercise. Acute phase inflammatory markers also rise during endurance exercise including ultramarathons and Ironman events. C Reactive Protein (CRP) is one of these acute phase inflammatory markers.

A cytokine involved in coordinating glucose homeostasis (eg glycogen resynthesis) and anti-inflammatory responses, IL-6, increases during endurance exercise. It has been measured 24 hours after an Ironman when levels are high and these remain high 5 days after the Ironman event.

The increase in inflammatory cytokines is, in part, due to the trauma caused to skeletal muscles during and after endurance exercise. The leading causes of trauma are eccentric muscle contractions, as in the long distance triathlon, (b) the impact of the extremities against the ground and (c) the number of repetitions of the same movement. The damage to the muscles may lead to a phenomenon of delayed onset muscle soreness (DOMS) that develops 24-48 hours post-endurance exercise that is accompanied by prolonged muscle strength loss, a reduced range of motion, and high levels of creatine kinase activity in the blood. Muscle swelling and DOMS is all consistent with muscle inflammation that is mediated by infiltrating neutrophils and macrophages that produce inflammatory cytokines but also prostaglandin E2 (PGE2), and this is a key mediator of pain 24-48 hours after an exercise session. Inflammatory activity within skeletal muscles can also be driven by local endothelial cells and smooth muscle cells. The cytokine inflammatory response persists for at least 5 days post-recovery as a low-grade systemic inflammatory response. For those that start training again, even at a level ⅕^{th} of the training prior to an Ironman race, the low-grade systemic inflammatory response can persist for longer.

The initial increase in pro-inflammatory cytokines is followed by elevated levels of anti-inflammatory cytokines. These anti-inflammatory cytokines such as IL-10 will act to dampen the pro-inflammatory response and prevent the overshooting of inflammation. Well-trained athletes are able to balance the acute exercise-induced inflammation by increasing their levels of IL-10. Increased IL-10 levels are associated with better performance.

Subjects that drop out of Ironman races due to self-reported fatigue symptoms have much higher pre-race CRP and IL-6 levels than those athletes that completed the race. The levels in the athletes that dropped out of the race were 316 to 1442% higher than the average pre-race concentration for those athletes that completed the race. Overtraining/prolonged training may lead to a prolonged inflammatory condition and lead to underperformance syndrome.

### Results

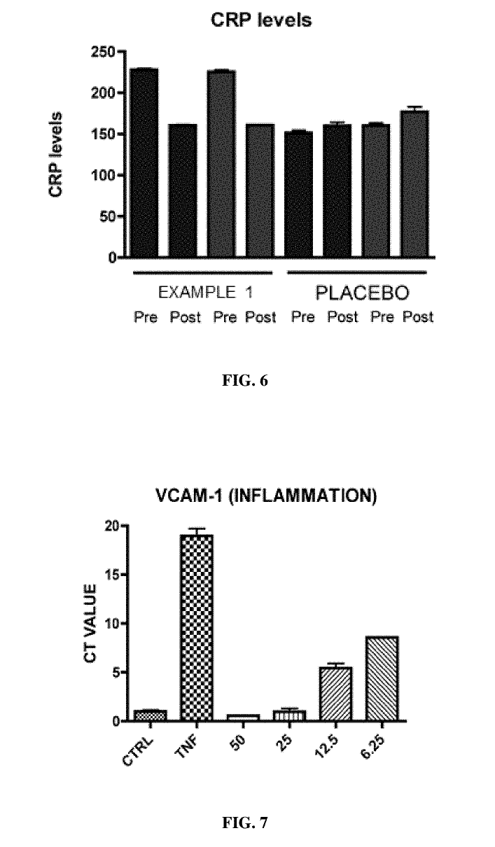

A laboratory study has shown that the composition of Example 1 can reduce plasma concentrations of the inflammatory marker, acute phase reactant, CRP (see FIG. 6—dark grey denotes runners and light grey denotes cyclists). Athletes were asked to complete a long run (15-20 km) or long ride (90 km+) on two separate occasions, 3-weeks apart. Blood sampling was performed prior to exercise and within 15 minutes post-exercise. On one of the occasions, the composition of Example 1 was ingested, prior to the training run or ride. On the other occasion a placebo supplement was ingested. The athletes were blinded to the supplement that they ingested each time.

ELISA tests showed that CRP levels were markedly decreased in the blood of the athletes post-training after the composition of Example 1, whereas there was no effect of the placebo supplement (slight increase in CRP levels after exercise). A decrease in CRP reflects a decrease in systemic inflammation caused by endurance exercise training. In turn, this may reduce muscle damage due to inflammation.

It was next tested if the composition of Example 1 has direct anti-inflammatory actions in human cells. To test this human coronary artery endothelial cells, the cells that line the arteries that supply blood to the tissues of the body, were exposed to the composition of Example 1 at concentrations equivalent to a 50-, 25-, 12.5- and 6.25-mL dose of the composition of Example 1. It was shown that the composition of Example 1 was able to suppress inflammation caused by the pro-inflammatory cytokine, TNFα, in human coronary artery endothelial cells as assessed by measuring an inflammatory marker in the cells called VCAM-1 (see FIG. 7).

In the above experiment, the composition of Example 1 was added directly to the cells from the bottle. However, in the body the composition of Example 1 is digested and components are absorbed into the blood stream where they pass through the liver and undergo type II metabolism. Therefore, the original molecules in the composition of Example 1 may be modified and it is these modified components that will cause the cells in the body to respond.

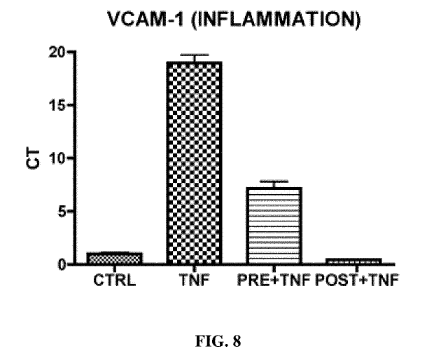

To test if the modified components of the composition of Example 1 are effective within the blood stream as anti-inflammatory molecules, subjects were asked to consume 100 ml of the composition of Example 1. Blood sampling was completed immediately before composition consumption and 3 hours after consumption. From the blood collected, the plasma was isolated. The plasma was then used to treat human coronary artery endothelial cells. Cells were treated for 24 hours with the plasma and then the cells were exposed to an inflammatory stimulus, pro-inflammatory cytokine, TNF-α. We then measured the extent of inflammation by monitoring VCAM-1 levels. Results are given in FIG. 8.

### Example 8

### Phase IIa Clinical Study

A phase IIa placebo controlled randomised clinical study has commenced with approximately 150 participants diagnosed with pre-diabetes. The clinical study is supplementing participants with a composition according to Example 1 [with or without metformin] or placebo for 12 weeks to ameliorate blood glucose levels as evidenced by an improvement in blood glucose levels according to the following outcomes:

### Primary:

Impaired Fasting Glucose (IFG) [measured by fasting glucose levels]

### Secondary:

Impaired Glucose Tolerance (IGT) [measured by Oral Glucose Tolerance Test (OGTT)]

hs-CRP

HbA1c levels

Safety (ELFTs, Urea, FBC)

HDL, LDL, TGs, Lipid profile

Quality of Life (SF-12v2 Questionnaire)

### Tertiary:

BP

Waist: hip ratio,

Weight

Dietary habits (3-day diet recall)

Physical activity (International Physical Activity Questionnaire—IPAQ)

Inclusion criteria:

Participants >18 years of age at time of entry on study; Male and Female, with Impaired fasting blood glucose
Initial online/telephone assessment will involve the Australian Type 2 Diabetes Risk Assessment Tool (AUSDRISK) questionnaire. Potential participants must have a score above 6 to qualify for next stage of assessment of meeting inclusion criteria.
Participants must demonstrate IFG—defined as fasting blood glucose 6.1-6.9 mmol/L
BMI≥25
Waist measurement: men≥102 cm; women≥88 cm
Cognitive ability to understand informed consent process and to give informed consent to the experimental treatment;
Participants agree to undergo venepuncture on multiple occasions
Participants agree to adhere to the study protocol, including not changing diet or exercise patterns over the 12 week study period.

Exclusion criteria:

Any clinically relevant abnormal findings, which in the opinion of the investigators/clinicians, may put the participant at risk of adverse events because of participation in the clinical trial including:
1) Physical examination; Clinical chemistry; Haematology; Urinalysis; Vital signs;
2) Diagnosis of Type II Diabetes Mellitus
3) Taking Anti-Obesity medications such as Orlistat
4) Taking oral blood glucose-lowering medications such as Sulfonylureas, Biguanides, Alpha-glucosidase inhibitors, Thiazolidinediones
5) Taking any dietary supplements such as herbal, multivitamin or mineral, probiotics, fish oils etc, prior to commencing study. However, if prescribed by their doctor they must NOT stop taking any form of dietary supplement. This will however make them ineligible to participate in the study.
6) Any allergies or past reactions to ingredients in treatment and placebo supplements, including Pinus pinaster or extracts thereof, papain (Carica papaya), aloe vera, honey, potassium sorbate and sodium benzoate
7. Alcohol abuse;
8. The use of any illicit drugs;
9. Pregnancy or nursing of an infant;
10. Commencing lifestyle interventions such as dietary changes and increasing exercise duration/intensity;
11. Any psychiatric disorders by history or examination that would prevent completion of the study or result in possible adverse events for the participant.

Once the eligibility criteria have been met, the participants will be randomized into two study arms namely, Group I and Group II. Group I to receive placebo and Group II to receive a composition of the invention. This study design was chosen as in an outpatient setting each participant can be considered to be a control subject in both arms of the study, thereby each participant acting also as their own control.

### Example 9

### Case Study 4: Patient ‘SG’ (Diabetes Type 1)

SG is a 23 year old female of normal weight diagnosed with type I diabetes on 9 Apr. 2015 after experiencing blurred vision, a huge thirst and fatigue. On 10 Apr. 2015, SG's blood glucose level was measured at 23.8 mmol/L.

SG was administered 100 mL per day of the composition of Example 1 in the mornings from 16 April 2015 to 15 May 2015. Administration of the composition of Example 1 ceased after 15 May 2015 for a few days, during which time SG's blood sugar levels began rising. Then, the composition of Example 1 was administered at a daily dose of 50 mL, taken as a single dose each morning. Blood glucose charts for SG are shown in Tables 5(a)-(k).

These charts show that SG's injected insulin levels are very low—which may be due to the composition of Example 1 helping the body to use the insulin efficiently, hence using less. SG's specialist commented that SG had attained a balance of blood sugar levels expected after 6 months, not 6 weeks. SG's blood sugar levels dropped whilst taking the composition of Example 1.

SG is now on an insulin pump and is maintaining the 50 mL per day of the composition of Example 1—which SG believes helps her feel normal, and is allowing her to dial in less insulin that is recommended for her carbohydrate intake.

TABLE 5(a)
WEEK BEGINNING: Apr. 6, 2015 (DATE)

| | | | | | | | | | | | | | | Remarks |
|---|---|---|---|---|---|---|---|---|---|---|---|---|---|---|
| | | | | | | | | | | | | | | Activity, illness, |
| Insulin Injections | | | | | | Monitoring Blood Glucose | | | | | | | | diet changes, time |
| | | Units given | | | | | | | | | | Before | | of hypos (noting |
| | Type of | | | | Before | Breakfast | | Lunch | | Dinner | | Supper | | blood glucose ad |
| | Insulin | Breakfast | Lunch | Dinner | Bed | Before | After | Before | After | Before | After | or Bed | Overnight | treatment) |
| Mon | | | | | | | | | | | | | | Eat |
| | | | | | | | | | | | | | | Blood Glucose |
| | | | | | | | | | | | | | | level |
| Tues | | | | | | | | | | | | | | |
| Wed | | | | | | | | | | | | | | Insulin |
| Thu | N R | 6 | 6 | 6 | | | | | 23.8 | | 19.8 | | | N R |
| | Lantus | | | 10 | 6 pm | | | | 17.1 | | | | | |
| Fri | N R | 6 | 8 | 8 | | 12.8 | 15.8 | | 11.2 | | 18.9 | | | eat |
| | Lantus | | | 12 | | | | | | | | | | |
| Sat | | 8 | 8 | 8 | | 10.4 | 14.8 | | 14.4 | | 8.7 | | | 2 hour |
| | | | | 12 | | | | | | | | | | |
| Sun | | 8 | | | | 7.2 | 7.0 | 11.3 | 10.3 | 16.1 | 11.0 | | | Test |

TABLE 5(b)
WEEK BEGINNING: Apr. 13, 2015 (DATE)

| | | | | | | | | | | | | | | Remarks |
|---|---|---|---|---|---|---|---|---|---|---|---|---|---|---|
| | | | | | | | | | | | | | | Activity, illness, |
| Insulin Injections | | | | | | Monitoring Blood Glucose | | | | | | | | diet changes, time |
| | | Units given | | | | | | | | | | Before | | of hypos (noting |
| | Type of | | | | Before | Breakfast | | Lunch | | Dinner | | Supper | | blood glucose and |
| | Insulin | Breakfast | Lunch | Dinner | Bed | Before | After | Before | After | Before | After | or Bed | Overnight | treatment) |
| Mon | N R | 8 | 8 | 8 | | 7.5 | 9.4 | 6.9 | (9.4) | | 6.3 | | | |
| | L | | | 12 | | | | | 4.4 | | | | | |
| Tues | N R | 8 | 6 | 8 | | 7.9 | 5.9 | | 8.5 | | 15.0 | | | |
| | L | | | 12 | | | | | | | | | | |
| Wed | N R | 8 | 8 | 8 | | 10.7 | 7.1 | 6.4 | 5.0 | 12.0 | 9.5 | 7.2 | | |
| | L | | | 12 | | | | | | | | | | |
| Thu | N R | 8 | 8 | 8 | | 6.5 | 6.7 | | 5.7 | 9.9 | 6.3 | 5.9 | | |
| | L | | | 12 | | | | | | | | | | |
| Fri | N R | 8 | 8 | 8 | | 8.0 | 9.3 | | 4.1 | | 9.5 | | | |
| | L | | | 12 | | | | | | | | | | |
| Sat | N R | 8 | 7 | 7 | | 6.5 | 3.4 | 5.3 | 4.4 | 9.4 | 2.7 | 9.2 | | |
| | L | | | 12 | | | | | | | 5.2 | | | |
| Sun | N R | 7 | 6 | 7 | | 6.6 | 2.9 | 7.2 | 10.1 | | 5.9 | 8.2 | | Walking |
| | L | | | 12 | | | 5.4 | | 8.6 | | | | | at 2:00 |

TABLE 5(c)
WEEK BEGINNING: Mar. 20, 2015 (DATE)

| | | | | | | | | | | | | | | Remarks |
|---|---|---|---|---|---|---|---|---|---|---|---|---|---|---|
| | | | | | | | | | | | | | | Activity, illness, |
| Insulin Injections | | | | | | Monitoring Blood Glucose | | | | | | | | diet changes, time |
| | | Units given | | | | | | | | | | Before | | of hypos (noting |
| | Type of | | | | Before | Breakfast | | Lunch | | Dinner | | Supper | | blood glucose and |
| | Insulin | Breakfast | Lunch | Dinner | Bed | Before | After | Before | After | Before | After | or Bed | Overnight | treatment) |
| Mon | | 7 | 5 | 4 | | 7.2 | 2.7 | 6.2 | 6.8 | | 13.9 | | | |
| | | | | 12 | | | 6.0 | | | | | | | |
| Tues | | 4 | 5 | 4 | | 5.6 | 5.0 | 5.0 | 11.8 | 8.9 | 6.9 | 7.7 | | |
| Wed | | 4 | 5 | 4 | | 5.5 | 8.4 | 6.8 | 6.2 | | 10.2 | 10.8 | | |
| Thu | | 4 | 5 | 4 | | 7.2 | 4.5 | 8.6 | 3.4 | | 9.3 | 10.2 | | |
| | | | | 12 | | | | | | | | | | |
| Fri | | 3 | 4 | 4 | | 6.7 | 8.4 | 8.9 | 6.3 | 9.3 | 11.2 | 9.2 | 6.5 | |
| Sat | | 3 | 5 | 4 | | 6.4 | 11.5 | 11.0 | 12.3 | | 7.7 | | 6.4 | |
| | | | | 12 | | | | | | | | | | |
| Sun | | 4 | 4 | 4 | | 6.5 | 6.0 | 5.0 | 4.9 | 6.7 | 13.1 | | | |
| | | | | 12 | | | | | | | | | | |

TABLE 5(d)
WEEK BEGINNING: Apr. 27, 2015 (DATE)

| | | | | | | | | | | | | | | Remarks |
|---|---|---|---|---|---|---|---|---|---|---|---|---|---|---|
| | | | | | | | | | | | | | | Activity, illness, |
| Insulin Injections | | | | | | Monitoring Blood Glucose | | | | | | | | diet changes, time |
| | | Units given | | | | | | | | | | Before | | of hypos (noting |
| | Type of | | | | Before | Breakfast | | Lunch | | Dinner | | Supper | | blood glucose and |
| | Insulin | Breakfast | Lunch | Dinner | Bed | Before | After | Before | After | Before | After | or Bed | Overnight | treatment) |
| Mon | | 4 | 4 | 4 | | 5.9 | 7.5 | | 3.8 | | 5.6 | 12.0 | 9.2 | |
| | | | | 12 | | | | | 7.2 | | | | | |
| Tues | | 4 | 4 | 4 | | 6.7 | 5.2 | | 6.2 | 10.1 | 13.8 | 10.8 | | |
| | | | | 12 | | | | | | | | | | |
| Wed | | 4 | 4 | 4 | | 7.1 | 5.3 | 7.6 | 7.4 | | 8.3 | | | |
| | | | | 12 | | | | | | | | | | |
| Thu | | 4 | 4 | 4 | | 5.9 | 8.0 | 6.0 | 9.9 | 9.3 | 8.0 | | | |
| | | | | 12 | | | | | | | | | | |
| Fri | | 4 | 4 | 4 | | 7.3 | 9.3 | 8.9 | 6.7 | 5.8 | 10.1 | | 8.0 | |
| | | | | 12 | | | | | | | | | | |
| Sat | | 4 | 4 | 5 | | 6.9 | 7.3 | | 10.0 | 9.4 | 9.00 | 10.3 | | |
| | | | | 12 | | | | | | | | | | |
| Sun | | 4 | 4 | 4 | | 5.3 | 7.0 | | 8.2 | | 7.3 | 10.0 | | |
| | | | | 12 | | | | | | | | | | |

TABLE 5(e)
WEEK BEGINNING: May 5, 2015 (DATE)

| | | | | | | | | | | | | | | Remarks |
|---|---|---|---|---|---|---|---|---|---|---|---|---|---|---|
| | | | | | | | | | | | | | | Activity, illness, |
| Insulin Injections | | | | | | Monitoring Blood Glucose | | | | | | | | diet changes, time |
| | | Units given | | | | | | | | | | Before | | of hypos (noting |
| | Type of | | | | Before | Breakfast | | Lunch | | Dinner | | Supper | | blood glucose and |
| | Insulin | Breakfast | Lunch | Dinner | Bed | Before | After | Before | After | Before | After | or Bed | Overnight | treatment) |
| Mon | | 4 | 4 | 5 | | 5.4 | 5.8 | | 6.0 | | 8.3 | 9.7 | | |
| | | | | 12 | | | | | | | | | | |
| Tues | | 4 | 4 | 4 | | 5.7 | 5.4 | | 4.6 | | 6.2 | 8.9 | | |
| | | | | 12 | | | | | | | | | | |
| Wed | | 4 | 4 | 4 | | 6.0 | 7.2 | | 5.7 | | 8.3 | 10.7 | | |
| | | | | 12 | | | | | | | | | | |
| Thu | | 4 | 5 | 4 | | 5.4 | 3.3 | 11.0 | 3.6 | | 4.6 | 7.0 | 7.1 | |
| | | | | 12 | | | 11.1 | | 6.0 | | | | | |
| Fri | | 4 | 3 | 3 | | 6.2 | 6.2 | 4.4 | 7.6 | | 10.7 | | 6.1 | |
| | | | | 12 | | | | | | | | | | |
| Sat | | 3 | 3 | 4 | | 5.9 | 6.2 | | 5.5 | | 9.5 | 9.9 | 6.8 | |
| | | | | 12 | | | | | | | | | | |
| Sun | | 4 | 4 | 5 | | 6.3 | 8.9 | | 6.4 | | 8.9 | 6.9 | | |
| | | | | 12 | | | | | | | | | | |

TABLE 5(f)
WEEK BEGINNING: May 11, 2015 (DATE)

| | | | | | | | | | | | | | | Remarks |
|---|---|---|---|---|---|---|---|---|---|---|---|---|---|---|
| | | | | | | | | | | | | | | Activity, illness, |
| Insulin Injections | | | | | | Monitoring Blood Glucose | | | | | | | | diet changes, time |
| | | Units given | | | | | | | | | | Before | | of hypos (noting |
| | Type of | | | | Before | Breakfast | | Lunch | | Dinner | | Supper | | blood glucose and |
| | Insulin | Breakfast | Lunch | Dinner | Bed | Before | After | Before | After | Before | After | or Bed | Overnight | treatment) |
| Mon | | 4 | 4 | 4 | | 4.8 | 6.8 | | 8.9 | | 7.4 | | | |
| | | | | 12 | | | | | | | | | | |
| Tues | | 3 | 4 | 4 | | 7.0 | 9.6 | | 8.0 | | 7.3 | | | |
| | | | | 12 | | | | | | | | | | |
| Wed | | 4 | 3 | 3 | | 6.3 | 10.3 | | 7.5 | | 11.0 | | | |
| | | | | 12 | | | | | | | | | | |
| Thu | | 4 | 4 | 4 | | 5.4 | 5.7 | | 7.4 | | 10.8 | | | |
| | | | | 12 | | | | | | | | | | |
| Fri | | 3 | 3 | 5 | | 5.1 | 6.4 | | 7.6 | | 9.7 | | | |
| | | | | 12 | | | | | | | | | | |
| Sat | | 3 | 4 | 3 | | 4.7 | 4.9 | | 6.8 | | 7.6 | | | |
| | | | | 12 | | | | | | | | | | |
| Sun | | 3 | 4 | 4 | | 5.3 | 6.5 | | 7.0 | | 9.3 | | | |
| | | | | 12 | | | | | | | | | | |

TABLE 5(g)
WEEK BEGINNING: May 18, 2015 (DATE)

| | | | | | | | | | | | | | | Remarks |
|---|---|---|---|---|---|---|---|---|---|---|---|---|---|---|
| | | | | | | | | | | | | | | Activity, illness, |
| Insulin Injections | | | | | | Monitoring Blood Glucose | | | | | | | | diet changes, time |
| | | Units given | | | | | | | | | | Before | | of hypos (noting |
| | Type of | | | | Before | Breakfast | | Lunch | | Dinner | | Supper | | blood glucose and |
| | Insulin | Breakfast | Lunch | Dinner | Bed | Before | After | Before | After | Before | After | or Bed | Overnight | treatment) |
| Mon | | 3 | 3 | 3 | | 5.7 | 5.5 | | 7.2 | | 7.0 | | | |
| | | | | 12 | | | | | | | | | | |
| Tues | | | | | | 5.4 | | | | | | | | |
| | | | | 12 | | | | | | | | | | |
| Wed | | | | | | | | | | | | | | |
| Thu | | | | | | | | | | | | | | |
| Fri | | | | | | | | | | | | | | |
| Sat | | | | | | | | | | | | | | |
| Sun | | | | | | | | | | | | | | |

TABLE 5(h)
WEEK BEGINNING: May 25, 2015 (DATE)

| | | | | | | | | | | | | | | Remarks |
|---|---|---|---|---|---|---|---|---|---|---|---|---|---|---|
| | | | | | | | | | | | | | | Activity, illness, |
| Insulin Injections | | | | | | Monitoring Blood Glucose | | | | | | | | diet changes, time |
| | | Units given | | | | | | | | | | Before | | of hypos (noting |
| | Type of | | | | Before | Breakfast | | Lunch | | Dinner | | Supper | | blood glucose and |
| | Insulin | Breakfast | Lunch | Dinner | Bed | Before | After | Before | After | Before | After | or Bed | Overnight | treatment) |
| Mon | | 3 | 3 | 4 | | 6.1 | 7.3 | | 9.0 | | 8.5 | | | |
| | | | | 12 | | | | | | | | | | |
| Tues | | 3 | 3 | 3 | | 5.9 | 10.6 | | 8.0 | | 12.3 | | | |
| | | | | 12 | | | | | | | | | | |
| Wed | | 3 | 3 | 3 | | 6.8 | 9.4 | | 7.7 | | 8.3 | | | |
| | | | | 12 | | | | | | | | | | |
| Thu | | 3 | 3 | 3 | | 5.3 | 7.9 | | 6.5 | | 8.0 | | 3.9 | |
| | | | | 12 | | | | | | | | | 7.4 | |
| Fri | | 3 | 3 | 3 | | 10.0 | 4.1 | | 8.3 | | 6.7 | | | |
| | | | | 10 | | | | | | | | | | |
| Sat | | 2 | 3 | 3 | | 7.0 | 8.2 | | 7.0 | | 8.3 | | | |
| | | | | 10 | | | | | | | | | | |
| Sun | | 2 | 3 | 2 | | 5.8 | 9.5 | 7.4 | 3.0 | | 6.9 | | | |
| | | | | 10 | | | | | 7.3 | | | | | |

TABLE 5(i)
WEEK BEGINNING: Jun. 1, 2015 (DATE)

| | | | | | | | | | | | | | | Remarks |
|---|---|---|---|---|---|---|---|---|---|---|---|---|---|---|
| | | | | | | | | | | | | | | Activity, illness, |
| Insulin Injections | | | | | | Monitoring Blood Glucose | | | | | | | | diet changes, time |
| | | Units given | | | | | | | | | | Before | | of hypos (noting |
| | Type of | | | | Before | Breakfast | | Lunch | | Dinner | | Supper | | blood glucose and |
| | Insulin | Breakfast | Lunch | Dinner | Bed | Before | After | Before | After | Before | After | or Bed | Overnight | treatment) |
| Mon | | 2 | 2 | 4 | | 7.3 | 6.3 | | 9.0 | | 7.3 | | | |
| | | | | 10 | | | | | | | | | | |
| Tues | | 2 | 3 | 3 | | 6.8 | 9.7 | | 9.8 | | 12.0 | | | |
| | | | | 10 | | | | | | | | | | |
| Wed | | 2 | 3 | 3 | | 6.4 | 10.3 | | 6.3 | | 5.6 | | | |
| | | | | 10 | | | | | | | | | | |
| Thu | | 2 | 2 | 3 | | 5.9 | 6.3 | 5.0 | 7.5 | | 11.3 | 10.0 | | |
| | | | | 10 | | | | | | | | | | |
| Fri | | 2 | 2 | 4 | | 7.5 | 8.0 | | 7.7 | | 6.2 | | | |
| | | | | 10 | | | | | | | | | | |
| Sat | | 2 | 3 | 4 | | 6.5 | 10.4 | | 7.0 | | 10.9 | | | |
| | | | | 10 | | | | | | | | | | |
| Sun | | 3 | 3 | 3 | | 7.4 | 7.0 | | 10.7 | | 10.6 | | | |
| | | | | 10 | | | | | | | | | | |

TABLE 5(j)
WEEK BEGINNING: Jun. 8, 2015 (DATE)

| | | | | | | | | | | | | | | Remarks |
|---|---|---|---|---|---|---|---|---|---|---|---|---|---|---|
| | | | | | | | | | | | | | | Activity, illness, |
| Insulin Injections | | | | | | Monitoring Blood Glucose | | | | | | | | diet changes, time |
| | | Units given | | | | | | | | | | Before | | of hypos (noting |
| | Type of | | | | Before | Breakfast | | Lunch | | Dinner | | Supper | | blood glucose and |
| | Insulin | Breakfast | Lunch | Dinner | Bed | Before | After | Before | After | Before | After | or Bed | Overnight | treatment) |
| Mon | | 2 | 4 | 3 | | 6.7 | 8.3 | | 5.3 | | 11.7 | | | |
| | | | | 10 | | | | | | | | | | |
| Tues | | 3 | 3 | 3 | | 7.4 | 10.0 | | 9.5 | | 12.8 | 11.9 | | |
| | | | | 10 | | | | | | | | | | |
| Wed | | 3 | 3 | 3 | | 6.5 | 8.5 | | 11.8 | | 5.7 | | | |
| | | | | 10 | | | | | | | | | | |
| Thu | | 3 | 3 | 3 | | 6.5 | 6.8 | | 7.5 | | 11.4 | | | |
| | | | | 10 | | | | | | | | | | |
| Fri | | 3 | 3 | 3 | | 7.7 | 7.0 | | 7.2 | | 8.1 | | | |
| | | | | 10 | | | | | | | | | | |
| Sat | | 3 | 3 | 3 | | 6.1 | 7.8 | | 9.0 | | 8.4 | | | |
| | | | | 10 | | | | | | | | | | |
| Sun | | 3 | 3 | 3 | | 7.6 | 11.3 | | 11.1 | | 13.2 | | | |
| | | | | 10 | | | | | | | | | | |

TABLE 5(k)
WEEK BEGINNING: Jun. 15, 2015 (DATE)

| | | | | | | | | | | | | | | Remarks |
|---|---|---|---|---|---|---|---|---|---|---|---|---|---|---|
| | | | | | | | | | | | | | | Activity, illness, |
| Insulin Injections | | | | | | Monitoring Blood Glucose | | | | | | | | diet changes, time |
| | | Units given | | | | | | | | | | Before | | of hypos (noting |
| | Type of | | | | Before | Breakfast | | Lunch | | Dinner | | Supper | | blood glucose and |
| | Insulin | Breakfast | Lunch | Dinner | Bed | Before | After | Before | After | Before | After | or Bed | Overnight | treatment) |
| | | | | | | | | | | | | | Z | |
| Mon | | 3 | 3 | 4 | | 6.6 | 9.9 | | 9.0 | | 13.9 | | | |
| | | | | 10 | | | | | | | | | | |
| Tues | | 3 | | | | 5.2 | 10.1 | | | | | | | |
| Wed | | | | | | | | | | | | | | |
| Thu | | | | | | | | | | | | | | |
| Fri | | | | | | | | | | | | | | |
| Sat | | | | | | | | | | | | | | |
| Sun | | | | | | | | | | | | | | |

### Example 10

### Case Study 5: Patient ‘A’ (High Cholesterol)

Patient A is a 51 year old male who has had high cholesterol for approximately 20 years, and who is reasonably fit and is not overweight.

Patient A has been taking 100 mL of the composition of Example 1 each day for a period of 3 weeks. The results of a blood test on 14 Jan. 2015 (prior to taking the composition of Example 1) and a blood test on 24 Jun. 2015 (after taking the composition of Example 1 for 3 weeks) gave the results in Table 6:

TABLE 6

| | Reading on | Previous reading | |
|---|---|---|---|
| Blood component | 24 Jun. 2015 | on 14 Jan. 2015 | Units |
| HDL | 1.3 | 1.1 | mmol/L |
| LDL | 5.6 | 5.8 | mmol/L |
| Total Chol/HDL Ratio | 5.8 | 6.9 | |
| Neutrophils | 47% | 41% | |
| ANC | 2.3 | 1.8 | |

The results in Table 6 show that the HDL (good cholesterol) levels in Patient A have improved whilst the LDL (bad cholesterol) levels have reduced, resulting in a significant change/reduction to Patient A's overall Total Cholesterol/HDL Ratio. Table 6 also shows an improvement in white blood cell count (neutrophils).

Patient A has also noticed that he is sleeping better and has more energy during the day whilst taking the composition of Example 1.

Patient A has not been taking Lipitor® or any other statins for over 18 months, as he has found the side effects of Lipitor® to be muscle aches and pain, tiredness, joint pain and a general feeling of not being well. His diet and exercise regime has not changed in the past 12 months.

### Example 11

### Case Study 6: Patient ‘PL’ (Diabetes Type 2)

Patient PL is a 63 year old male diagnosed with Diabetes Type 2 in Mar. 2012. As part of his treatment for Diabetes 2, PL has been taking metformin since diagnosed. He is of thin build and is not overweight.

PL commenced taking a daily dose of 80 to 100 mL of the composition of Example 1 on 1 May 2015 and ceased taking metformin at the same time. The results of PL's blood tests (measuring HBA1c) are given in Table 7.

TABLE 7

| | | Test result | |
|---|---|---|---|
| | Test result | (HBA1c (IFCC); | Test result |
| Date | (HBA1c; %) | mmol/mol) | (eAG; mmol/L) |
| 26 Jun. 2012 | 6.3 H | — | 7.4 |
| 26 Mar. 2013 | 6.5 H | 47 H | 7.8 |
| 1 Jul. 2015 | 6.0 H | 42 H | 7.0 |

As shown in Table 7, PL's recent blood test (1 Jul. 2015) indicates an HBA1c level of 6.0 after taking the composition of Example 1 for a period of 2 months, which is down from the previous test level of 6.5 (on 26 Mar. 2013). eAG levels are also down after taking the composition of Example 1. PL has advised that blood glucose self-monitoring has seen his BGL reduce from a general level of approximately 8.0 mmol/L prior to taking the composition of Example 1 to approximately 6.0 mmol/L now (i.e., after taking the composition of Example 1).

### Example 12

### Case Study 7: Patient ‘TG’ (Lupus)

Patient TG is a male suffering from lupus for the past 18 years. Recently, he began taking the composition of Example 1 at a dosage of 100 mL per day in the morning. After approximately 2-3 weeks (having taken 1.5 L), the sub cutaneous lesions on TG's face and scalp for years have reportedly disappeared and have not returned.

TG also reports episodes of clarity that last for hours, better immune system function, and generally feeling better as a result of taking the composition of Example 1.

### Example 13

### Case Study 8: Patient ‘MS’ (Emphysema)

Patient MS is a male patient diagnosed with emphysema, who has been taking 100 mL of the composition of Example 1 daily for a period of a few years prior to December 2014 since having surgery to remove most of his right lung.

Patient MS suffered from major shortness of breath and found it difficult to climb stairs after removal of most of his right lung. However, after taking the composition of Example 1 for one month, he reported that he was breathing more easily and experiencing less difficulty climbing stairs. After three months of taking the composition of Example 1, Patient MS reported that his X-rays and MR1 results showed no signs of emphysema. He reported that his doctor was amazed at this result and this type of lung disease does not usually go away and instead usually gets worse over time.

Patient MS also reported that his overall health has been exceptional since taking the composition of Example 1, and that he has not had a cold, flu or other common ailment for years. He also notes that there has been no decrease in his lung capacity since taking the composition of Example 1.

### Example 14

### Case Study 9: Patient JW (High Cholesterol and High Blood Pressure)

Patient JW is a female patient suffering from heart problems, high cholesterol, high blood pressure, and breathing problems. She began taking 50 mL of the composition of Example 1 daily in the morning about 1 year prior to December 2014. In December 2014, she reported that her high cholesterol had reduced and that she had increased energy as a result of taking the composition of Example 1.

### Example 15

### Case Study 10: Patient ‘S’ (Psoriasis)

Patient S is a female patient suffering from psoriasis of the scalp. After taking the composition of Example 1 for a few days, Patient S reported that the psoriasis on her scalp improved.

### Example 16

### Case Study 11: Patient ‘M’ (Psoriasis)

Patient M is a female patient suffering from plaque psoriasis. After taking 30 mL of the composition of Example 1 daily each morning for around 7 weeks, Patient M reported reduced redness with her flaky skin and less plaque showing, particularly on her knees.

1. A composition comprising Pinus pinaster stem bark extract, papain, and Aloe vera extract.

2. A composition comprising a therapeutically effective amount of Pinus pinaster stem bark extract, papain, and Aloe vera extract.

3. The composition according to claim 1, wherein the Aloe vera extract is Aloe vera leaf extract.

4. The composition according to claim 2, wherein the therapeutically effective amount of Pinus pinaster stem bark extract corresponds to a daily adult human dosage of from about 60 mg to about 1500 mg Pinus pinaster stem bark extract;
the therapeutically effective amount of papain corresponds to a daily adult human dosage of from about 30 mg to about 1200 mg papain; and
the therapeutically effective amount of Aloe vera extract corresponds to a daily adult human dosage of from about 15 mg to about 600 mg Aloe vera extract.

5. The composition according to any one of claim 2, wherein the therapeutically effective amount of Pinus pinaster stem bark extract corresponds to a daily adult human dosage of about 260 mg;
the therapeutically effective amount of papain corresponds to a daily adult human dosage of about 240 mg; and
the therapeutically effective amount of Aloe vera extract corresponds to a daily adult human dosage of about 175 mg.

6. The composition according to claim 1, wherein the composition further comprises an acid.

7. The composition according to claim 6, wherein the acid is acetic acid.

8. The composition according to claim 6, wherein the composition comprises acid in a concentration effective to adjust the pH of the composition to between about 3.2 and about 4.2.

9. The composition according to claim 1, wherein the composition further comprises honey.

10. The composition according to claims 1, wherein the composition comprises one or more additives selected from the group consisting of: a preservative, a flavouring agent, a salt, and a pigment.

11. The composition according to claim 1, wherein the composition comprises a salt, and wherein the salt is sodium chloride.

12. The composition according to claim 1, further comprising an additional component selected from the group consisting of: an omega-3 fatty acid, a phytonutrient, a source of protein, an amino acid, an antioxidant, a vitamin, a mineral, a plant extract, and mixtures thereof.

13. The composition according to claim 1, wherein the composition is synergistic.

14. The composition according to claim 1, wherein the composition is adapted for oral administration.

15. The composition according to claim 14, wherein the composition is in a form selected from the group consisting of: a tablet, a capsule, a chewable tablet, a soft gel capsule, a sachet, a powder, granules, a liquid, a syrup, a liquid suspension, an emulsion, a solution, and combinations thereof.

16. The composition according to claim 1, wherein the composition is an aqueous composition.

17. The composition according to claim 1, wherein the composition has a pH of about 3.7.

18. The composition according to claim 16, wherein the Pinus pinaster stem bark extract is present at a concentration of from about 2 mg/mL to about 10 mg/mL.

19. The composition according to claim 18, wherein the Pinus pinaster stem bark extract is present at a concentration of about 2.6 mg/mL.

20. The composition according to claim 16, wherein the papain is present at a concentration of from about 1 mg/mL to about 5 mg/mL.

21. The composition according to claim 20, wherein the papain is present at a concentration of about 2.4 mg/mL.

22. The composition according to claim 16, wherein the Aloe vera extract is present at a concentration of from about 0.5 mg/mL to about 4 mg/mL.

23. The composition according to any claim 16, wherein the Aloe vera extract is present at a concentration of about 1.75 mg/mL.

24. The composition according to claim 16, wherein the composition further comprises glacial acetic acid at a concentration of from about 1 mg/mL to about 8 mg/mL.

25. The composition according to claim 24, wherein the glacial acetic acid is present at a concentration of about 2 mg/mL.

26. The composition according to claim 15, wherein the composition comprises honey at a concentration of from about 50 mg/mL to about 200 mg/mL.

27. The composition according to claim 26, wherein the honey is present at a concentration of about 100 mg/mL.

28. An aqueous composition comprising about 2.6 mg/mL Pinus pinaster stem bark extract, about 2A mg/mL papain, and about 1.75 mg/mL Aloe vera extract.

29. The composition according to claim 28, further comprising about 100 mg/mL honey.

30. The composition according to claim 28, further comprising about 2 mg/mL glacial acetic acid.

31. An aqueous composition comprising:
2.40 mg/mL papain;
2.60 mg/mL Pinus pinaster stem bark extract;
1.75 mg/mL Aloe vera inner leaf juice;
100.00 mg/mL honey;
2.00 mg/mL glacial acetic acid;
3.00 mg/mL sodium chloride;
400 μg/mL potassium sorbate;
400 μg/mL sodium benzoate;
10.00 mg/mL wildberry flavour UA71225; and
800 /mL anthocyanin extract.

32. An aqueous composition comprising papain, Pinus pinaster stem bark extract, Aloe vera inner leaf juice, honey, glacial acetic acid, sodium chloride, potassium sorbate, sodium benzoate, wildberry flavour UA71225 and anthocyanin extract in a ratio of
papain:dry Pinus pinaster stem bark extract:dry Aloe vera inner leaf juice:honey:glacial acetic acid:sodium chloride:potassium sorbate:sodium benzoate:wildberry flavour UA71225:anthocyanin extract
of 2.40:2.60:1.75:100:2.00:3.00:0.40:0.40:10.00:0.80 by weight.

33. An aqueous composition comprising papain, dry Pinus pinaster stem bark extract, dry Aloe vera inner leaf juice, honey, glacial acetic acid, and sodium chloride in a ratio of
papain:dry Pinus pinaster stem bark extract:dry Aloe vera inner leaf juice:honey:glacial acetic acid:sodium chloride
of 2.40:2.60:1.75:100:2.00:3.00 by weight.

34. An aqueous composition comprising papain, Pinus pinaster stem bark extract, and Aloe vera inner leaf juice in a ratio of
papain:dry Pinus pinaster stem, bark extract:dry Aloe vera inner leaf juice of 2.40:2.60:1.75 by weight.

35. A method of treating or preventing elevated blood glucose, comprising administering to a subject in need thereof an effective amount of a composition according to claim 1.

36. A method of preventing or delaying the onset of type 2 diabetes mellitus (T2DM), comprising administering to a subject in need thereof an effective amount of a composition according to claim 1.

37. A method of treating or preventing pre-diabetes, comprising administering to a subject in need thereof an effective amount of a composition according to claim 1.

38. A method of reducing inflammation, comprising administering to a subject in need thereof an effective amount of a composition according to claim 1.

39. A method of decreasing cytokine-induced inflammation, comprising administering to a subject in need thereof an effective amount of a composition according to claim 1.

40. A method of decreasing an oxidative stress response, comprising administering to a subject in need thereof an effective amount of a composition according to claim 1.

41. A method of treating or preventing a disease characterised by elevated levels of cytokine-induced inflammation, comprising administering to a subject in need thereof an effective amount of a composition according to any claim 1.

42. A method of treating or preventing a disease characterised by elevated levels of oxidative stress, comprising administering to a subject in need thereof an effective amount of a composition according to claim 1.

43. The method of claim 41, wherein the disease is atherosclerosis, liver inflammation associated with type 2 diabetes and obesity, endometriosis, or osteoarthritis.

44. A method of reducing one or more side effects of a diabetes medication selected from the group consisting of metformin, sulfonylureas, rneglitinides, thiazolidinediones, DPP-4 inhibitors, GLP-1 receptor agonists, SGLT2 inhibitors, and insulin therapy, the method comprising administering to a subject in need thereof an effective amount of a composition according to claim 1.

45. The method of claim 44, wherein the side effect is abdominal or stomach discomfort, decreased appetite, or diarrhoea.

46. A method of treating or preventing a dysfunction associated with excess blood glucose, comprising administering to a subject in need thereof an effective amount of a composition according to claim 1.

47. The method of claim 46, wherein the dysfunction is selected from the group consisting of: increased thirst and hunger, frequent urination, fatigue/lethargy blurry vision, sleepiness, and muscle weakness.

48. The method of claim 35, wherein the composition is administered in combination with metformin or another blood glucose lowering medication.

49. A method of improving organ function comprising administering to a subject in need thereof an effective amount of a composition according to claim 1.

50. A method of lowering blood cholesterol, comprising administering to a subject in need thereof an effective amount of a composition according to claim 1.

51. The method of claim 50, wherein the blood cholesterol is selected from the group consisting of: low density lipoproteins (LDL), high density lipoproteins (HDL), trigylcerides and total cholesterol.

52. A method of treating an autoimmune disease, comprising administering to a subject in need thereof an effective amount of a composition according to claim 1.

53. The method of claim 52, wherein the autoimmune disease is selected from the group consisting of: lupus, arthritis, psoriasis, and type I diabetes.

54. The method according to claim 35, wherein the effective amount of the composition corresponds to:
a daily adult human dosage of from about 60 mg to about 1500 mg Pinus pinaster stem bark extract;
a daily adult human dosage of from about 30 mg to about 1200 mg papain; and
a daily adult human dosage of from about 15 mg to about 600 mg Aloe vera extract.

55. The method according to claim 35, wherein the effective amount of the composition corresponds to:
a daily adult human dosage of about 260 mg Pinus pinaster stem bark extract;
a daily adult human dosage of about 240 mg papain; and
a daily adult human dosage of about 175 mg Aloe vera extract.

56. The method according to claim 54, wherein the daily adult human dosage of Pinus pinaster stem bark extract, papain and Aloe vera extract is provided to the subject in a single dose.

57. The method according to claim 54, wherein the daily adult human dosage of Pinus pinaster stem bark extract, pepsin and Aloe vera extract is provided to the subject in multiple doses.

58. Use of a composition according to claim 1 for the manufacture of a medicament for:
treating or preventing elevated blood glucose; or
preventing or delaying the onset of type 2 diabetes mellitus (T2DM); or
treating or preventing pre-diabetes; or
reducing inflammation; or
decreasing cytokine-induced inflammation; or
decreasing an oxidative stress response; or
treating or preventing a disease characterised by elevated levels of cytokine-induced inflammation; or
treating or preventing a disease characterised by elevated levels of oxidative stress; or
reducing one or more side effects of a diabetes medication selected from the group consisting of metformin, sulfonylureas, meglitinides, thiazolidinediones. DPP-4 inhibitors, GLP-1 receptor agonists, SGLT2 inhibitors, and insulin therapy; or
treating or preventing a dysfunction associated with excess blood glucose; or
improving organ function; or
lowering blood cholesterol;
in a subject.

59. The method of claim 35, wherein the subject is selected from the group consisting of the elderly, those with a medical condition of pre-diabetes or at risk of developing pre-diabetes, and combinations thereof.

60. The use of claim 58, wherein the subject is taking metformin or another blood glucose lowering medication.

61. A method of manufacturing an aqueous composition, said method comprising:
combining Pinus pinaster stem bark extract, papain, and Aloe vera extract in water.

62. The method of claim 61, wherein the method further comprises:
adding an acid.

63. The method of claim 62, wherein the acid is glacial acetic acid.

64. The method of claim 61, further comprising:
adding honey.
